(19) United States
(12) Patent Application Publication
(10) Pub. No.: US 2024/0067955 A1
(43) Pub. Date: Feb. 29, 2024

(54) METHODS FOR HIGH THROUGHPUT SCREENING OF CHIMERIC ANTIGEN RECEPTORS

(71) Applicant: CODING BIO LIMITED, Oxford, Oxfordshire (GB)

(72) Inventors: Simon BORNSCHEIN, Oxford, Oxfordshire (GB); Youlia LAMPI, Oxford, Oxfordshire (GB)

(21) Appl. No.: 18/261,425

(22) PCT Filed: Jan. 20, 2022
(86) PCT No.: PCT/GB2022/050158

§ 371 Date: Jul. 13, 2023

(30) Foreign Application Priority Data
Jan. 20, 2021 (GB) 2100754.7
Nov. 16, 2021 (GB) 2116519.6

(51) Int. Cl. C12N 15/10; G01N 33/68

(52) U.S. Cl. CPC C12N 15/1037; C12N 15/1055; G01N 33/6845

(57) ABSTRACT

A method for high-throughput screening of a chimeric antigen receptor (CAR) cell library is provided comprising the steps of (a) providing a recognition sequence library, a hinge region sequence library, a transmembrane sequence library and an intracellular domain sequence library; (b) preparing a CAR library; (c) preparing a CAR-cell library by introduction to, and expression of, the plurality of CAR sequences of the CAR library in one or more cells or a cell line; (d) screening the CAR-cell library in an assay; (e) evaluating the at least one function of each member of the CAR-cell library; (f) obtaining one or more sequences of one or more CARs expressed in the CAR-cell library and linking the obtained sequence(s) to the at least one function of the members of the CAR-cell library; g) identifying and selecting the or each sequence based on function. Methods of preparing the CAR library and a CAR-cell library and uses thereof are also provided.

### FIELD OF THE INVENTION

The invention is in the field of chimeric antigen receptors (CARs), and in particular methods for the pre-clinical evaluation and subsequent selection of CARs based on required functionality.

### BACKGROUND OF THE INVENTION

Chimeric antigen receptors (CARs) are synthetic, engineered, membrane-bound receptors that are typically used to target surface molecules on other cells. CARs generally comprise an extra-cellular portion having an antigen binding domain, for example, a single chain variable fragment (scFv) that engages a target, a hinge region, a trans-membrane domain and an intracellular domain that is responsible for downstream signalling.

CARs have found use in the treatment of disease, particularly oncology, when present on a T-cell. Two CAR-T therapies, both directed to the B cell antigen CD19 have recently been approved by the FDA for use in the US.

To date, CARs are typically developed by first identifying and optimising an antibody to the target. This involves a variety of means, including preliminary antibody screening or panning, followed by characterization of any hits including sequencing the key recognition sequences. Once suitable antibodies are identified, typically based on affinity and/or specificity for the target alone, they may undergo additional levels of antibody optimization, such as through affinity maturation. At this point a small number will then be selected for further development in which the antigen binding regions (e.g. CDRs) are incorporated into a CAR and tested for functionality such as biological activity, toxicity, and cytokine production. If suitable functionality in the CAR is not achieved, the process must be repeated.

An inherent limitation in this process is that the selection of lead-clinical candidates is based mainly on criteria that select for and optimise good monoclonal antibodies, but not for the desired clinical end product, which is the CAR.

For a given target, the number of selected antibodies taken forward to incorporation in a CAR allowed for current cloning and testing Is around a maximum of ten CARs but more often only between two and five at a time. This number is low mainly because it is a resource intensive and labour-intensive process involving deconvoluting and sequencing the antibody, identifying the key sequences for recognition (CDRs), incorporating these into a suitable CAR scaffold and then manufacturing of viral particles, transduction of cells and assessing their functionality in a manual, low throughput manner. With a low number of CARs progressed, the chance of success of any one of those candidates is low meaning the process may need to be repeated, sometimes more than once. Each iteration of this process takes many months and, typically around 1-2 years.

The prior art shows that most researchers found 100+ scFvs that target the antigen of interest but were only able to advance around 2-5 of these to a screen when incorporated into a CAR.

Examples of the standard CAR production processes are summarised in:
Preclinical Evaluation of Allogeneic CART Cells Targeting BCMA for the Treatment of Multiple Myeloma”, Molecular Therapy, 2019.
US 2019/0161553 A1
US 20170283504 A1

It is evident that this protracted approach also has a disproportionately high risk of failure as a functional antibody does not necessarily align with CAR functionality. Highly functional antibodies can be poor performers when incorporated into a CAR due to many reasons, for example tonic signalling of the CAR (which is scFv dependent), epitope accessibility to CAR vs. mAb and biochemical stability of the fusion receptor. Ghorashian et al. (Nature Medicine, 2019) showed that lowering CAR affinity can result in increased serial killing and improved therapeutic performance. Contrary, other reports (e.g. Hudecek et al. Clinical Cancer Research, 2013) demonstrated that CARs based on high affinity scFvs showed greater anti-tumor potency compared to CARs with lower affinity scFvs. Thus, affinity of the scFvs is not universal and depends on interconnected factors such as antigen densities on target cells, CAR expression levels, and binding epitope location. None of these can be predicted based on antibody characteristics.

Other relevant literature:
“CAR-T design: Elements and their synergistic function”, EBioMedicine, VOLUME 58, 102931, August 2020. (review of the challenges in CAR engineering and optimization)
Ghorashian, S., Kramer, A. M., Onuoha, S., Wright, G., Bartram, J., Richardson, R., et al (2019). Enhanced CAR T cell expansion and prolonged persistence in pediatric patients with ALL treated with a low-affinity CD19 CAR. Nature Medicine. https://doi.org/10.1038/s41591-019-0549-5 (lowering CAR affinity increases serial killing and therapeutic performance)
Liu, X., Jiang, S., Fang, C., Yang, S., Olalere, D., Pequignot, E. C., et al. (2015). Affinity-tuned ErbB2 or EGFR chimeric antigen receptor T cells exhibit an increased therapeutic index against tumors in mice. Cancer Research. https://doi.org/10.1158/0008-5472.CAN-15-0159. (lowering CAR affinity increases therapeutic index against tumors)
Hudecek, M., Lupo-Stanghellini, M. T., Kosasih, P. L., Sommermeyer, D., Jensen, M. C., Rader, C., & Riddell, S. R. (2013). Receptor affinity and extracellular domain modifications affect tumor recognition by ROR1-specific chimeric antigen receptor T cells. Clinical Cancer Research. https://doi.org/10.1158/1078-0432.CCR-13-0330 (high affinity CARs have increased anti-tumor efficacy)
Zhang, Z., Jiang, D., Yang, H., He, Z., Liu, X., Qin, W., et al. (2019). Modified CAR T cells targeting membrane-proximal epitope of mesothelin enhances the antitumor function against large solid tumor. Cell Death and Disease. https://doi.org/10.1038/s41419-019-1711-1. (Structural as well as functional aspects of the target epitope affect CAR functionality and should be included in design considerations for CARs).
James, S. E., Greenberg, P. D., Jensen, M. C., Lin, Y., Wang, J., Till, B. G., et al, Press, 0. W. (2008). Antigen Sensitivity of CD22-Specific Chimeric TCR Is Modulated by Target Epitope Distance from the Cell Membrane. The Journal of Immunology. https://doi.org/10.4049/jimmunol.180.10.7028. (A membrane-distal epitope of CD22 was found to have weaker signaling, lower lytic efficiency, and defective degranulation compared to CARs binding to a membrane-proximal epitope)
Di Roberto, R. B. et al, “A functional Screening strategy for Engineering Chimeric Antigen Receptors with Reduced On-Target, Off-Tumor Activation; Molecular Therapy; Vol. 28(12), December 20 pp 2564-2576 describes the production of a ‘library’ of CARs and their screening. However, the method of preparing the library is limited by the process of preparing cells with “protoCARs”, i.e. CARs that do not have a full recognition site, instead having a partial recognition site and a locus for gene editing to incorporate a single point of variability in the recognition sequence (scFv). The paper relates solely to the optimisation of a known recognition sequence for certain benefits and fails to disclose a production of a CAR library or high throughput screening of a CAR library with multiple points of diversity in the recognition sequence and/or CAR scaffold.

Liu Delong et al., J. Hematology & Oncology, vol. 12, 2019, pp 1 describes a modular approach to the design and preparation of CARs. This approach requires specific synthesis of modules, or domains of the CAR with specific binding regions for attachment to a neighbouring domain. The method is also not compatible with a screening approach in a pooled manner, as antibody modules could bind to cells at random, due to them being soluble. It also results in screening of CARs that are inherently different to those CARs that may be used in therapy which would require a single continuous CAR structure, expressed from a single continuous CAR sequence to avoid any risk of separation of the CAR modules.

WO 2015/123642 describes the preparation of CAR sequences from three components provided as libraries of vectors by random homologous recombination. No means by which the sequence of the CAR or component parts of a CAR can be linked to a functional result of a cell in a suitable high-throughput manner is provided. No means by which extrapolation of the result to refine and develop promising CAR cell therapies has been described.

CAR functionality is determined by the coordinated activities of each of the five key domains in the CAR structure (recognition domain, the hinge region domain, the transmembrane region domain, and the intracellular domain comprising a stimulatory and optional co-stimulatory domain. There therefore remains a need to identify methods and systems which the function of a CAR can be evaluated based on high-throughput screening of the CAR itself, and not on a surrogate, for example, associated antibody activity. There also remains a need to develop a method of preparing and screening a CAR library in which multiple sites of variation of the CAR (for example each CDR in the scFv or other recognition sequence, and/or the individual parts of the scaffold of the CAR) can be evaluated in a high-throughput manner to promote efficient identification of CAR clinical candidates.

### SUMMARY OF THE INVENTION

In a first aspect, the invention provides a method for high-throughput screening of a chimeric antigen receptor (CAR)-expressing cell library, the method comprising the steps of:
a) providing:
   i) a recognition sequence library, wherein the recognition sequence library comprises a one or more recognition sequences, wherein each recognition sequence encodes one or more antigen binding domains;
   ii) a hinge region sequence library, wherein the hinge region sequence library comprises one or more hinge region sequences, wherein each hinge region sequence encodes a CAR hinge region domain;
   iii) a transmembrane sequence library, wherein the transmembrane sequence library comprises one or more transmembrane sequences, wherein each transmembrane sequence encodes a CAR transmembrane domain; and
   iv) an intracellular domain sequence library, wherein the intracellular domain sequence library comprises one or more intracellular domain sequences, wherein each intracellular domain sequence encodes one or more intracellular domains;
b) preparing a CAR library from the combination of:
   i) a recognition sequence from the recognition sequence library;
   ii) a hinge region sequence from the hinge region sequence library;
   iii) a transmembrane sequence from the transmembrane sequence library; and
   iv) an intracellular domain sequence from the intracellular domain sequence library;
      wherein the CAR library comprises a plurality of CAR sequences, each of the CAR sequences comprising one recognition sequence, one hinge region sequence, one transmembrane domain sequence and one intracellular domain sequence, and wherein each CAR sequence is a single continuous sequence that encodes a chimeric antigen receptor;
c) preparing a CAR-cell library by introduction to, and expression of, the plurality of CAR sequences of the CAR library in one or more cells or a cell line so a plurality of CARs encoded by the plurality of CAR sequences are expressed on a surface of the one or more cells or a cell line;
d) screening the CAR-cell library in an assay that reports at least one function of each member of the CAR-cell library;
e) evaluating, or assessing, the at least one function of each member of the CAR-cell library;
f) obtaining one or more sequences, or a part thereof, of one or more of the plurality of CARs expressed in the CAR-cell library and linking the obtained sequence(s), or the part thereof, to the at least one function of one or more members of the CAR-cell library;
g) identifying and selecting the or each sequence that is responsible for a desired function of a member of the CAR-cell library.

In embodiments, the recognition sequence library, the hinge region sequence library, the transmembrane sequence library and intracellular domain sequence library of step (a)(i) to (iv) may be provided as distinct separate libraries. In alternative embodiments, each of these libraries may be pre-combined with one or more other libraries in any manner such that the preparing of a CAR-library in step (b) provides a sequence for a CAR.

In a second aspect, the invention provides a method for high-throughput screening a chimeric antigen receptor (CAR) library, the method comprising the steps of:
a) providing a recognition sequence library, wherein the recognition sequence library comprises one or more, suitably a plurality of recognition sequences, wherein each recognition sequence encodes for one or more antigen binding domains;
b) preparing a CAR library from the combination of a recognition sequence library and a CAR scaffold, wherein the CAR scaffold comprises a hinge region sequence, a transmembrane domain sequence and an intracellular domain sequence, suitably wherein the CAR library comprises a plurality of CAR sequences, each of the CAR sequences comprising one of the one or more recognition sequences, the hinge region sequence, the transmembrane domain sequence and the intracellular domain sequence, and wherein each CAR sequence is a single continuous sequence that encodes a chimeric antigen receptor;
c) preparing a CAR-cell library wherein each CAR sequence of the CAR library is expressed as a CAR on the cell surface of one or more cells or a cell line, suitably the CAR-cell library is prepared by transfecting the or each CAR sequence of the CAR library into one or more cells or a cell line so the CAR is expressed on the cell surface;
d) screening the CAR-cell library in an assay that reports at least one function of the or each member of the CAR-cell library;
e) evaluating, or assessing, the at least one function of the or each member of the CAR-cell library;
f) obtaining one or more sequences, or a part thereof, of one or more of the plurality of CARs expressed in the CAR-cell library and linking the obtained sequence(s), or the part thereof, to the at least one function of one or more members of the CAR-cell library;
g) identifying and selecting the or each sequence that is responsible for a desired function of a member of the CAR-cell library.

Any manner of combining the plurality of recognition sequences with the other components comprised in the CAR scaffold is contemplated. In an embodiment, the CAR scaffold is provided as a single unitary sequence for combination with the recognition sequence library, i.e. the remainder of the CAR is provided as a single sequence to which the recognition sequence is incorporated to provide a sequence for a complete CAR. In alternative embodiments, the CAR scaffold is assembled from its component parts (i.e. the hinge region sequence, the transmembrane domain sequence and the intracellular domain sequence) separately, prior to or after combination with the recognition sequence library.

In embodiments, the CAR scaffold is provided as separate components when combined with the recognition sequence (for example in a pooled combination of libraries of one or more, suitably a plurality, of sequences of one or more of: the hinge region sequence, the transmembrane domain sequence and the intracellular domain sequence). In embodiments, the recognition sequence is combined with one component, suitably the hinge region sequence, prior to combination with other components in an appropriate manner to provide a single continuous sequence encoding for a complete CAR. Such embodiments would encompass addition of further components of the CAR sequence in a sequential and/or parallel manner using individual sequences or libraries of a plurality of sequences, of the added components in an appropriate order. Alternatively, in embodiments, addition of the additional components is after one or more of the components has been combined, i.e. pre-combined sections of the CAR sequence formed of one or more sequences of the added components, such sections being formed by combination of individual sequences or libraries of sequences of the or each component(s). All embodiments described above provide a CAR library of a plurality of CAR sequences, each sequence encoding for a CAR. In embodiments, each CAR sequence in the CAR library is formed of a recognition sequence and a CAR scaffold comprising a hinge region sequence, a transmembrane domain sequence and an intracellular domain sequence.

The following paragraphs refer to embodiments that are equally applicable to the first or second aspect of the invention.

In embodiments, the sequence, or part thereof, of each of the plurality of CARs in the cells in the CAR-cell library are identified and linked to the function of an individual cell within the CAR-cell library.

In embodiments, the method comprises deconvolution of the structure of the CAR. Suitably deconvolution is from pooled or mixed cells in the CAR-cell library. Suitably deconvolution comprises assigning a sequence to one or more CARs expressed on cells on any given cell, suitably a cell identified as having the desired function in the CAR-cell library. The method of deconvolution may be by any suitable means. Suitably the method of deconvolution is through spatial positioning of the cells (i.e. in a well plate), through identification markers, such as barcode sequences that report the sequence of one or more, suitably all, components of the CAR sequence. In embodiments deconvolution of the sequence of the CAR from pooled or mixed cells in the CAR-cell library is through long-read next generation sequencing methods such as Oxford Nanopore™ sequencing, PacBio™ sequencing, Loop Genomics LoopSeg™ or similar.

In embodiments, the CAR-cell library has at least two points of diversity in the structure of each CAR represented therein. Suitably, the at least two points of diversity are selected from the group consisting of: one or more recognition sequences, suitably antigen binding domains; the hinge domain; the transmembrane domain; the intracellular domain and combinations thereof.

In embodiments, the CAR library comprises one or more CAR sequences or vectors, each CAR sequence or CAR vector encoding a CAR. Suitably each CAR sequence in the CAR library encodes for a different CAR. In embodiments that make use of CAR vectors, introduction of the or each CAR in the CAR library in step (c) comprises transfection, transduction or electroporation of the CAR vector into the one or more cells or the cell line of the CAR-cell library.

In embodiments, the method is used for high-throughput screening of more than 10 CARs expressed in the CAR-cell library. Suitably, the method screens more than 20, 30, 40, 50, 60, 70, 80, 90, 100, 150, 200 or more CARs.

In embodiments, each cell in the CAR-cell library expresses an individual CAR.

In embodiments, the at least one function of each member of the CAR-cell library is selected from the group consisting of: affinity binding to a target; downstream signalling of the CAR intracellular domain; modulation of protein and/or RNA expression.

In embodiments, the function in step (d) is a measurable activation of the cells in the assay of step (d) to provide activated cells. Suitably, activated cells may be identified based on up-regulation of: activation markers; cytokines; and/or introduced reporters driving expression of a reporter gene. Suitably, activated cells may be identified by a method selected from: FACS sorting and magnetic enrichment or a combination thereof. In embodiments, the CAR sequence of an activated cell is identified using one or more methods selected from the group consisting of: long-read sequencing; next generation sequencing (NGS), Sanger sequencing.

In embodiments, screening of the library is performed in a manner selected from the group consisting of: simultaneously; in parallel; pooled: batchwise and any combination thereof. Suitably, the function in step (d) comprises modulation of the RNA expression of the cell in the CAR-cell library. Suitably, evaluation of the modulation in gene expression comprises analysis of RNA expression of one or more genes. In embodiments, the analysis of RNA expression of one or more genes uses single cell RNA expression measurement techniques.

In embodiments, the reporting of the modulation in gene expression in step (d) comprises comparison of single cell RNA expression data of activated screened cells against the same, control cells that have not been subject to screening. Suitably, the comparison uses RNA-seq techniques to analyse the cellular transcriptome of each cell in the CAR-cell library.

In embodiments, expressed mRNA of each cell in the CAR-cell library is associated with a unique identifier. Suitably, mRNA of the CAR expressed in the cell is associated with the same unique identifier as the other mRNA from that cell such that the unique identifier may be used to associate the CAR to the mRNA expression of an individual cell.

In embodiments, the unique identifier is a barcode sequence. Suitably, the barcode sequence is a DNA barcode sequence attached to a cDNA complimentary to the expressed RNA.

In embodiments, the unique identified allows linking of the sequence of the or each individual CAR, or part thereof, to the function of an individual cell within the CAR-cell library in step (g).

In embodiments, the CAR sequence may be identified using one or more methods selected from the group consisting of: long-read sequencing; next generation sequencing (NGS), and Sanger sequencing. Suitably, the one or more CARs of interest identified in step (g) provide novel CAR structures for a given target. Suitably, the one or more CARs of interest identified in step (g) are used for the design of further iterations of the method of screening of the first or second aspect of the invention.

In embodiments, machine learning algorithms are used to identify and select CAR sequences or parts thereof for further iterations of screening, Suitably, one or more CAR sequences in the CAR library further comprises a co-stimulatory domain sequence.

In embodiments, the recognition sequence, the hinge region sequence, the transmembrane sequence. the intracellular domain sequence, and the CAR sequence are each a nucleic acid sequence. Suitably, the nucleic acid sequence is a DNA sequence.

In embodiments, screening of the library is performed in a high-throughput manner selected from the group consisting of: simultaneously; in parallel; pooled: batchwise and any combination thereof.

In embodiments, at least steps (d) to (g) of the screening method of the first or second aspect aspect or may be completed within 24 hours, suitably 18 hours, suitably 12 hours. In embodiments, in step (f), an individual sequence, or part thereof, is obtained and linked to the at least one function of an individual member of the CAR-cell library in which the individual sequence is expressed.

In a third aspect, the invention provides a CAR library for use in the high-throughput method of the first aspect or the second aspect of the invention, wherein the CAR library comprises a plurality of sequences encoding for a CAR, the CAR comprising a recognition sequence from the recognition sequence library, a hinge region, a transmembrane domain sequence and an intracellular domain sequence, and wherein each CAR sequence is a single continuous sequence that encodes a chimeric antigen receptor.

In embodiments, the CAR library has at least two points of diversity or variability in the sequence of each CAR represented therein. Suitably, the at least two points of diversity are selected from the group consisting of: one or more antigen binding domains; the hinge domain; the transmembrane domain; and the intracellular domain. Suitably, the one or more antigen binding domains comprises at least two or more antigen binding domains.

In embodiments, the CAR library comprises sequences for more than 10 CARs. Suitably, the CAR library comprises sequences for more than 20, 30, 40, 50, 60, 70, 80, 90, 100, 150, 200 or more CARs.

In embodiments, the CAR-library is provided in a format suitable for introduction into cells or a cell line to form a CAR-cell library. Suitably, each CAR sequence in the CAR library is provided as a vector suitable for introduction and/or expression (suitably, transfection; transduction; and electroporation) of the CAR sequence into cells.

In embodiments, the CAR sequences of the CAR-library are provided in a format selected from: plated with CAR sequences split into separate wells or compartments of a plate; pooled where more than one CAR sequence is in a single well or compartment of a plate; and combinations thereof.

In a fourth aspect, the invention provides a method of preparing a CAR library of the third aspect of the invention, the method comprising the steps (a) and (b) of the first aspect of the invention or the second aspect of the invention.

As above, in embodiments of the second aspect of the invention, the CAR scaffold may be provided as a single unitary sequence to be combined with the plurality of recognition sequences in the recognition sequence library, or the CAR scaffold may be provided as individual components either before or after combination with the plurality of recognition sequences in the recognition sequence library. The individual components of the CAR scaffold may be provided as individual sequences or each component, region or domain, or as one or more libraries of sequences for each individual component, region or domain.

In a fifth aspect, the invention provides a CAR-cell library for use in the parallel high-throughput screening method of the first aspect or the second aspect of the invention, wherein the CAR-cell library comprises a plurality of cells, each cell having at least one CAR expressed on its surface, wherein each CAR comprises a recognition sequence domain, a hinge region domain, a transmembrane domain and an intracellular domain.

In embodiments, the CAR-cell library has at least two points of diversity or variability in the structure of each CAR represented therein. Suitably, the at least two points of diversity are selected from the group consisting of: one or more antigen binding domains; the hinge domain; the transmembrane domain; the intracellular domain and combinations thereof.

In embodiments, the CAR-cell library comprises more than 10 cells, each cell having at least one CAR expressed on its surface. Suitably, the CAR-cell library comprises more than 20, 30, 40, 50, 60, 70, 80, 90, 100, 150, 200 or more CARs.

In embodiments, a single CAR is expressed on the surface of a single cell.

In embodiments, the cell is a T-cell. Suitably, the T-cell is a primary human T-cell.

In embodiments, the CAR-cell library is provided in a format suitable for screening. Suitably, the cells of the CAR-cell library are provided in a format selected from: plated with one or more cells split into separate wells or compartments of a plate; pooled where more than one cell is in a single well or compartment of a plate; and combinations thereof.

In a sixth aspect, the invention provides a method of preparing a CAR-cell library of the fifth aspect of the invention, wherein the method comprises steps (a), (b) and (c) of the first aspect of the invention or steps (a), (b) and (c) of the second aspect of the invention. As above, in embodiments of the second aspect, the CAR scaffold may be provided as a single unitary sequence to be combined with the plurality of recognition sequences in the recognition sequence library, or the CAR scaffold may be provided as individual components either before or after combination with the plurality of recognition sequences in the recognition sequence library. The individual components of the CAR scaffold may be provided as individual sequences or each component, region or domain, or as one or more libraries of sequences for each individual component, region or domain.

In a seventh aspect, the invention provides use of the method of the first aspect or the second aspect for the identification of CARs having a desired function.

In an eighth aspect, the invention provides use of the CAR library of the third aspect to prepare a CAR-cell library for screening.

In a ninth aspect, the invention provides use of a CAR-cell-library of the fifth aspect for screening the function of a plurality of CARs. Suitably, the screening is performed in a high-throughput manner selected from the group consisting of: simultaneously, in parallel, pooled, batchwise. Suitably, the plurality of CARs is more than 10 CARs.

### BRIEF DESCRIPTION OF THE FIGURES

FIG. 1: High-throughput CAR screening. A multitude of recognition sequences/targeting moieties (e.g. scFv, VHH), linker domains, hinge domains, transmembrane domains and intracellular signalling domains (co-stimulatory, stimulatory and/or inhibitory) are cloned and assembled together in a pooled manner to form a single continuous CAR sequence construct. The resulting library of CAR constructs is then introduced into/expressed in cells and assessed for functionality and further characterized. Computational methods (e.g. machine learning) can be applied to select a functional receptor as a prospective or lead candidate.

FIG. 2: High-throughput CAR screening. A first multitude of a recognition sequences/targeting moieties (e.g. ScFv, VHH) is combined with a second multitude of targeting moieties against the same or different target and a multitude of linker domains, hinge domains, transmembrane domains and intracellular signalling domains (co-stimulatory, stimulatory and/or inhibitory) are cloned and assembled together in a pooled manner. The resulting library of CAR constructs is then introduced into cells and assessed for functionality and further characterized. Computational methods (e.g. machine learning) can be applied to select a functional receptor as lead candidate.

FIG. 3: Next generation sequencing of assembled CAR library from PCR amplicons. CAR library, targeting BCMA, was sequenced through next generation sequencing on an Oxford Nanopore™ MinION™. Frequency of scFvs (color coded for each individual scFv) in the different frameworks was assessed.

FIG. 4: Confirmation of BCMA binding and surface expression of CAR-cell library. BCMA-CAR-T library cells were stained with BCMA-Fc fusion protein (TNFRSF17-Fc, R&D systems) (left) or a negative control protein (TNFRSF13B-Fc, R&D systems) (right).

FIG. 5: Assessing CAR affinity/avidity on CAR-T cell library. T cells were transduced with the BCMA CAR library and the resulting CAR-T cell library assessed for BCMA binding by flow cytometry (left). rBCMA-Fc protein was titrated on the cells in order to determine frequency of CARs with a given affinity/avidity on a population scale. Percentage of BCMA-positive cells relative to protein concentration used (right)

FIG. 6: Sorting for high avidity/affinity CAR constructs: CAR-T cell library, reactive against BCMA, was generated and exposed to rBCMA-Fc protein and assessed by flow cytometry before (left) and after sorting for PE-positive cells.

FIG. 7: Sorting for activated CAR constructs: CAR-T cell library, reactive against BCMA, was generated and exposed to BCMA-expressing HeLa cell line. After 24 h, activated CAR-T cells were sorted for CD69-positivity by f magnetic bead selection. Left: CD69 enriched cells; right: CD69-negative fraction.

FIG. 8: Enrichment of transduced cells. BCMA CAR-T library was assessed for tCD34 expression (left) and selected for transduced, tCD34-positive cells through anti-CD34 magnetic bead selection (right).

FIG. 9: Single cell sequencing of CAR-T cell library. Single cell sequencing data were analyzed and dimensional reduction (UMAP) of the single-cell sequencing data and their functional clusters is shown. Each greyscale shade represents an assigned cluster.

FIG. 10. Tonic activation signature. Tonic activation signature was identified in cells, not exposed to target cells. Degree of tonic signaling is scored by greyscale shade score.

FIG. 11. CAR activation signature. CAR-T activation signature was defined based on unsupervised clustering and genesets, downstream of CAR activation pathway were further defined. Greyscale shade scale indicates degree of activation score

FIG. 12. Location of CARs with favourable clinical outcomes in UMAP plot and identified and selected as CARs of interest.

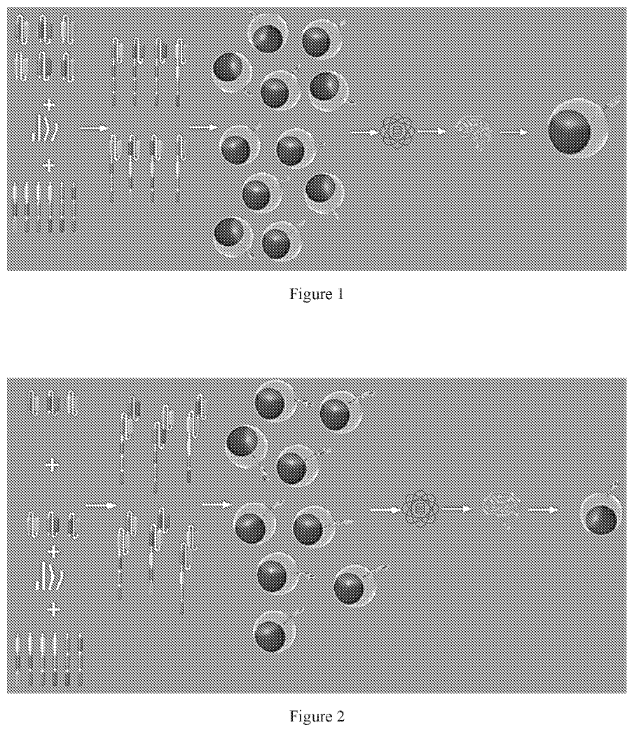

### DETAILED DESCRIPTION OF THE INVENTION

All references cited herein are incorporated by reference in their entirety. Unless otherwise defined, all technical and scientific terms used herein have the same meaning as commonly understood by one of ordinary skill in the art to which this invention belongs.

Prior to further setting forth the invention, a number of definitions are provided that will assist in the understanding of the invention.

The articles “a”, “an” and “the” are used to refer to one or to more than one (i.e. to at least one) of the grammatical object of the article.

As used herein, the term “comprising” means any of the recited elements are necessarily included and other elements may optionally be included as well. “Consisting essentially of” means any recited elements are necessarily included, elements which would materially affect the basic and novel characteristics of the listed elements are excluded, and other elements may optionally be included. “Consisting of” means that all elements other than those listed are excluded. Embodiments defined by each of these terms are within the scope of this invention.

Unless specifically defined herein, all terms used herein have the same meaning as they would to one skilled in the art of the present invention. Practitioners are particularly directed to Green and Sambrook, Molecular Cloning: A Laboratory Manual, 4th ed., Cold Spring Harbor Laboratory Press, New York (2012); and Ausubel et al., Current Protocols in Molecular Biology (up to Supplement 114), John Wiley & Sons, New York (2016), for definitions and terms of the art. The definitions provided herein should not be construed to have a scope less than understood by a person of ordinary skill in the art.

As used herein the term “antigen binding domain” refers to a peptide sequence that is intended or able to bind a target of interest. All types of antigen binding domains are encompassed by the present invention. Examples of some antigen binding domains are scFvs VHH single domain antibodies or nanobodies, and antigen binding fragments. An “scFv” of “single chain variable fragment” is a type of antigen binding domain. Typically, an scFv is a fusion of the variable regions of the heavy (VH) and light chains (VI) of an antibody for a given target connected by a short linker. Antigen binding domains may comprise “CDRs” or “complementarity determining regions” which are predominantly responsible for target binding. On a typical antibody, multiple CDRs exist and may be selected or varied independently to achieve multiple points of diversity. A ‘recognition sequence’ refers to the nucleic acid sequence encoding one or more antigen binding domains.

A “transmembrane domain” or “TM domain” as used herein is any membrane-spanning protein domain. Suitably, the TM domain in a CAR is derived from a known transmembrane protein sequence. However, it can also be artificially designed. A ‘transmembrane sequence’ refers to the nucleic acid sequence encoding a transmembrane domain.

The term “signaling domain” or “intracellular domain” or “intracellular signaling domain” as used herein refers to a moiety that can transmit a signal in a cell, for example an immune cell. The signaling domain typically comprises a domain derived from a receptor that signals by itself in immune cells, such as the T Cell Receptor (TCR) complex or the Fc receptor or DAP10/DAP12 receptors. Additionally, it may contain a costimulatory domain (i.e. a domain derived from a receptor that is required in addition to the TCR to obtain full activation, or the full spectrum of the signal in case of inhibitory costimulatory domains, of T cells). The costimulatory domain can be from an activating costimulatory receptor or from an inhibitory costimulatory receptor. An Intracellular domain sequence′ refers to the nucleic acid sequence encoding an intracellular signaling domain.

“Antibody” refers to all isotypes of immunoglobulins (IgG, IgA, IgE, IgM, IgD, and IgY) including various monomeric, polymeric and chimeric forms, unless otherwise specified.

Specifically encompassed by the term “antibody” are polyclonal antibodies, monoclonal antibodies (mAbs), single domain antibodies, human (FHVH) or heavy-chain antibodies found in camelids (VHH) and antibody-like polypeptides, such as chimeric antibodies and humanized antibodies. “Antigen-binding fragments” are any proteinaceous structure that may exhibit binding affinity for a particular antigen. Antigen-binding fragments include those provided by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation diabodies and single-chain molecules as well as Fab, F(ab′)2, Fc, Fabc, and Fv molecules, single chain (sc) antibodies, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the VL, VH, CL and CHI domains, or a monovalent antibody as described in WO2007059782, bivalent fragments comprising two Fab fragments linked by a disulfide bridge at the hinge region, a Fd fragment consisting essentially of the V.sub.H and C.sub.HI domains; a Fv fragment consisting essentially of the VL and VH domains of a single arm of an antibody, a dAb fragment (Ward et al., Nature 341, 544-546 (1989)), which consists essentially of a VH domain and also called domain antibodies (Holt et al; Trends Biotechnol. 2003 November; 21(11):484-90); camelid or nanobodies (Revets et al; Expert Opin Biol Ther. 2005 January; 5(1): 111-24); an isolated complementarity determining region (CDR), and the like. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds. Antigen-binding fragments may be recombinantly produced or produced by enzymatic or chemical cleavage of intact antibodies. The phrase “an antibody or antigen-binding fragment thereof may be used to denote that a given antigen-binding fragment incorporates one or more amino acid segments of the antibody referred to in the phrase.

“Specific binding” or “immunospecific binding” or derivatives thereof when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a KD of less than about 1×10⁻⁸ M, as measured by a surface plasmon resonance assay or a cell binding assay. Phrases such as “[antigen]-specific” antibody (e.g., BCMA-specific antibody) are meant to convey that the recited antibody specifically binds the recited antigen.

The phrase “nucleic acid molecule” synonymously referred to as “nucleotides” or “nucleic acids” or “polynucleotide” refers to any polyribonucleotide or polydeoxyribonucleotide, which may be unmodified RNA or DNA or modified RNA or DNA. Nucleic acid molecules include, without limitation single- and double-stranded DNA, DNA that is a mixture of single- and double-stranded regions, single-and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

There are various means by which a nucleic acid sequence may be inserted into a genome, including but not limited to plasmid or vector transfection, transposition and genome editing. All are contemplated for use in the present invention. A “vector” is a replicon, such as plasmid, phage, cosmid, or virus in which another nucleic acid segment may be operably inserted so as to bring about the replication or expression of the segment. A “transposon” or “transposable elements” are DNA sequences that can change their position within a genome. “Genome editing” refers to the ability to edit the genome to insert the required sequence, for example using CRISPR-Cas9 genome editing technology.

A “clone” is a population of cells derived from a single cell or common ancestor by mitosis.

A “cell line” is a clone of a primary cell that is capable of stable growth in vitro for many generations. In some examples provided herein, cells are transformed by transfecting the cells with DNA.

The terms “express” and “produce” are used synonymously herein and refer to the biosynthesis of a gene product. These terms encompass the transcription of a gene into RNA. These terms also encompass translation of RNA into one or more polypeptides, and further encompass all naturally occurring post-transcriptional and post-translational modifications.

A “point of diversity” of a CAR or CAR library or CAR-cell library as used herein means a component or region in the structure of a CAR that may be varied to modulate or optimise its function. A point of diversity may comprise one or more regions of the binding moiety or recognition sequence, and/or the choice or adaptation of one or more components of the CAR scaffold, such as the hinge region, a transmembrane portion and an intracellular domain.

The term “subject” refers to human and non-human animals, including all vertebrates, e.g., mammals and non-mammals, such as non-human primates, mice, rabbits, sheep, goats, dogs, cats, horses, cows, chickens, amphibians, and reptiles. In most particular embodiments of the described methods, the subject is a human.

The terms “treating” or “treatment” refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

As used herein, the term “high-throughput screening” refers to any assay or screening methodology that allows for a higher rate of screening than would be achieved by traditional or previous state of the art techniques. Typically, high-throughput screening enables automation to prepare, screen and/or evaluate libraries of test samples in parallel, reproducibly and rapidly. High-throughput screening can also make use of combinatorial or pooled or mixed sample screening strategies, with associated deconvolution of hits. In the context of CAR high throughput screening, the number of samples for test may be of any size larger than that typically used in prior art non-high-throughput methods. For example, the number of samples may be more than 10. Suitably the number of samples may be more than 11, 12, 13, 14, 15, 16, 17, 18, 19, 20, 30, 40, 50, 100, 200 or more.

As used herein, the term “chimeric antigen receptor” or “CAR” refers to transmembrane receptor that has been engineered to target or bind to a non-native substrate or antigen. In this way the intracellular signalling of the receptor may triggered by binding of a non-native substrate or antigen. Typically, the term CAR refers to a chimeric receptor (i.e. a receptor composed of two or more parts from different sources) that has at least a binding moiety or recognition sequence with a specificity for a target such as an antigen or protein and an intracellular signaling domain that can invoke a signal in the cell in which the CAR is expressed (e.g. a CD3 zeta chain). In embodiments, a “chimeric antigen receptor” or “CAR” is formed of at least three domains: an extracellular antigen binding domain, a transmembrane domain and an intramolecular domain. A hinge domain between the antigen binding domain and the transmembrane domain is often used to improve recognition of the target. A co-stimulatory domain may optionally be present in the intracellular domain to modulate the response. To be functional, the domains of the CAR must be ordered correctly. CARs are often used on T-cells (to produce “CAR T-cells”) to effect recognition and an appropriate intracellular response which both binds the T-cell to a target cell and triggers the innate an immune response of the T-cell, typically lysis of the target cell. Such cells have found use in therapy.

As used herein, the term “recognition sequence library” refers to a set of one or more antigen binding domain sequences or recognition sequences that may be used for cloning into a CAR construct (wherein a CAR construct comprises all components required for a functioning CAR including a one or more antigen binding domains, a hinge domain, a transmembrane domain and an intracellular domain) to prepare a CAR library. An “scFv library” is a recognition sequence library formed of scFv recognition sequences from antibodies. In embodiments, the number of recognition sequences present in the recognition sequence library may be more than 1 or more than 2. Suitably the number of hinge region sequences present in the hinge region sequence library may be more than 3, 4, 5, 6, 7, 8, 9, 10, 11, 12, 13, 14, 15, 16, 17, 18, 19, 20, 30, 40, 50, 100, 200 or more.

As used herein, the term “hinge region sequence library” refers to a set of hinge region sequences that may be used for cloning into a CAR construct (wherein a CAR construct comprises all components required for a functioning CAR including a one or more antigen binding domains, a hinge domain, a transmembrane domain and an intracellular domain) to prepare a CAR library. In embodiments, the number of hinge region sequences present in the hinge region sequence library may be more than 1 or more than 2. Suitably the number of hinge region sequences present in the hinge region sequence library may be more than 3, 4, 5, 6, 7, 8, 9, 10, 11, 12, 13, 14, 15, 16, 17, 18, 19, 20, 30, 40, 50, 100, 200 or more.

As used herein, the term “transmembrane sequence library” refers to a set of transmembrane sequences that may be used for cloning into a CAR construct (wherein a CAR construct comprises all components required for a functioning CAR including a one or more antigen binding domains, a hinge domain, a transmembrane domain and an intracellular domain) to prepare a CAR library. In embodiments, the number of hinge region sequences present in the hinge region sequence library may be more than 1 or more than 2. Suitably the number of hinge region sequences present in the hinge region sequence library may be more than 3, 4, 5, 6, 7, 8, 9, 10, 11, 12, 13, 14, 15, 16, 17, 18, 19, 20, 30, 40, 50, 100, 200 or more.

As used herein, the term “intracellular domain sequence library” refers to a set of intracellular domain sequences that may be used for cloning into a CAR construct (wherein a CAR construct comprises all components required for a functioning CAR including a one or more antigen binding domains, a hinge domain, a transmembrane domain and an intracellular domain) to prepare a CAR library. In embodiments, the number of intracellular domain sequences present in the intracellular domain sequence library may be more than 1 or more than 2. Suitably the number of hinge region sequences present in the hinge region sequence library may be more than 3, 4, 5, 6, 7, 8, 9, 10, 11, 12, 13, 14, 15, 16, 17, 18, 19, 20, 30, 40, 50, 100, 200 or more.

As used herein, the term “CAR scaffold” refers to the part of the CAR sequence that comprises the components, parts, modules, domains of the CAR excluding the recognition sequence that form the sequence of a CAR in a CAR library. The CAR scaffold is formed of the sequences of the individual components, or groups thereof, and combined with the recognition sequence to form a sequence encoding a CAR in any suitable manner, for example, sequentially, convergently, with the recognition sequence being incorporated at any suitable point, i.e. the recognition sequence may be joined to a single unitary sequence of the CAR scaffold, or may be joined to a component part of the CAR scaffold initially and the full CAR sequence of the recognition sequence and scaffold completed subsequently. The component parts of the CAR scaffold may derive from single sequences leading to one or a small number of CAR scaffolds for addition to a recognition sequence, or the component parts of the CAR scaffold may derive from libraries of one or more of the individual components leading to a CAR scaffold library comprising a plurality of sequences. A CAR scaffold library may be formed by combination, suitably in a combinatorial, or directed, manner of the individual components or component libraries.

As used herein, the term “CAR library” refers to a set of sequences encoding for a functional CAR structure, including a recognition domain, such as an scFv, a hinge domain, a transmembrane domain and an intracellular domain. In embodiments, the number of CARs present in the CAR library may be more than 10. Suitably the number of CARs present in the CAR library may be more than 11, 12, 13, 14, 15, 16, 17, 18, 19, 20, 30, 40, 50, 100, 200 or more. The term ‘CAR library’ may also be used interchangeably to refer to the plasmid or vector, or otherwise modified sequence of the sequence(s) encoding for a functional CAR structure.

As used herein, the term “CAR-cell library” refers to a collection or set of cells expressing CARs on their surface. Each cell in the CAR-cell library may express a single CAR (i.e. express only one CAR encoded by a single sequence), or a single cell in the CAR-cell library may express two or more CARs, each encoded by a different sequence. Suitably, each cell in the CAR-cell library expresses a single CAR or all CARs present in the library. In embodiments, the number of CARs present in the CAR-cell library may be more than 10. Suitably the number of CARs present in the CAR-cell library may be more than 11, 12, 13, 14, 15, 16, 17, 18, 19, 20, 30, 40, 50, 100, 200 or more.

As used herein, the term “next generation sequencing” of “NGS” refers to a catch-all term used to describe a number of different modern sequencing technologies. These technologies allow for sequencing of DNA and RNA much more quickly and cheaply than the previously used Sanger sequencing. Examples are Solex™ sequencing by Illumina™, Roche 454™ sequencing, Ion Torrent™ sequencing and nanopore based sequencing methods (e.g. Oxford Nanopore Technologies™ Grid ION™).

The invention relates, in one aspect, to a method for high throughput screening that is able to prepare and rapidly evaluate a large number of CARs for a desired function, and optionally use this to select the best clinical candidates. In embodiments, the invention relates to a method of identifying and/or selecting a CAR by high throughput processing, or screening, of libraries of CARs.

To date, the development of a CAR, for example for CAR-T therapies, has followed the accepted process of (1) selecting an antigen target; (2) identifying an antibody for the given antigen target, typically this is by some form of antibody enrichment procedure such as screening or panning a phage display, immunisation or yeast display library, for example, and optionally further optimising the antibody to develop the antigen binding properties; (3) characterising the antibody specificity, affinity and to identify the sequence of the recognition sequences (CDRs); (4) incorporate the selected ScFv or the CDR sequence into a scFv of a CAR, along with a choice of transmembrane domain and intracellular signalling domain; (5) evaluate the properties of the CAR in in-vitro and then a clinical context.

This process suffers from a number of significant drawbacks.

Firstly, the protracted process of isolating and characterising antibodies from the initial screen in step (2) is both labour and resource intensive. This limits the number of CARs that can be prepared and evaluated from the initial screen.

Secondly, isolating and characterising antibodies brings forward costs and effort to the front end of the process so that considerable screening effort is spent on understanding and characterising the antibody, despite the fact that this is not the desired clinical product.

Thirdly, antibody activity in vitro does not always translate to equivalent CAR activity in a clinical context. This can mean a given antibody with promising baseline activity is progressed for evaluation as a CAR where it can fail to show the desired properties in a clinical context. Indeed, this is one of the known areas of failure in conventional CAR development processes which prioritise antigen binding affinity at the early stage over clinical efficacy of the ultimate CAR T cell product.

Fourthly, the ability to vary multiple potential points of diversity in the CAR is severely limited by the number of CARs produced, and the stepwise process in which CAR development is conducted. The recognition domain, including each individual CDR in the CAR recognition sequence, and/or other parts of the CAR scaffold (hinge region. Transmembrane domain and intracellular domain) may have an impact on CAR function that is difficult to predict and must be tested.

The present inventors have appreciated that the accepted process for the identification of a CAR clinical candidate is flawed and should be rationalised so that resources are focussed primarily on the diversity of the CAR library and identification of the CARs that are able to demonstrate promising activity in a functional assay relevant to a clinical context. In embodiments of the invention, the process of pre-selection of recognition sequences or antibodies that show affinity for the target, or at least fully characterising the antibodies, enriched through phage display (or immunization or yeast display or another method), is dispensed with. Instead, the key target or antigen recognition sequences of any antibody meeting an established affinity threshold are directly cloned, in a high throughput manner, into a CAR scaffold which is then expressed in a suitable cell line or cell, for example, a primary cell (e.g. human T cells, NK cells, regulatory T cells).

The straightforward nature of the process of the present invention, along with the simplicity and familiarity of the steps involved, allows for ease of manufacture of a CAR-cell library. The process also allows for a drastic increase in the diversity of the CARs to be tested, through the increased number and potential for diversity at multiple points in the recognition sequence and/or within the CAR scaffold structure. This combinatorial approach allows a CAR-cell library prepared in accordance with the present invention to cover more of the potential active space of the CAR-cell library leading to a genuine high-throughput approach to screening for CAR clinical candidates based on the functional evaluation of the CAR in a clinical context.

This advancement of the present invention means the costly and time-consuming isolation and pre-selection, or at least the characterisation, of the antibodies is bypassed thereby allowing for the generation of larger and more diverse CAR libraries. Once the library of CARs is prepared then it may be screened against many targets or in other assays in order to identify one or more CAR candidates in a clinically-relevant assay.

Once identified, only those CARs that show promise need be further evaluated meaning expensive and time-consuming evaluation and deconvolution steps are pushed to the end of the process when a CAR clinical candidate has been identified, and more confidence can be placed on its success.

In embodiments, the process of generating a CAR library and selecting a CAR clinical candidate comprises one or more of the following steps:

Selection of Biological Target

A target is selected based on appropriate knowledge and understanding from the literature, databases, etc. The target may be any suitable biological entity that may be recognised as an antigen by an antibody, or an scFv or other suitable binding moiety of a CAR. Suitably, the target can be selected from the group consisting of: proteins; peptides, MHC presented peptides, MHC-like presented peptides. The target may comprise any one of the following:
Cell surface proteins—receptors; receptor complexes; glycoproteins; cytoskeletal proteins; ion channels; and transmembrane proteins
Cell surface oligosaccharides—N-linked or O-linked glycosylation
Microbial antigens—viral, bacterial, protozoan or fungal antigens

Pre-Selection of Suitable Recognition Sequences for the CAR Library—Optional

Potential target-recognition sequences for use as the binding moiety portion of the CAR may be randomly selected, rationally designed, computationally designed, for example with AI or machine learning techniques or identified and/or selected by any suitable means. In embodiments where there is some form of pre-selection of the binding moiety, potential recognition sequences are identified by antigen affinity binding techniques where a suitable recognition sequence is identified by affinity binding to the biological target. In some embodiments, potential recognition sequences are identified by immunizing an animal. In embodiments, the scFv portion for incorporation into a CAR library is identified from the corresponding antibody. Alternatively, recognition sequence libraries may be obtained commercially, or generated randomly or based on other selection approaches.

Antibody Enrichment

For embodiments of the present invention that rely on pre-selection of putative scFv sequences by antibody binding, the means of identifying antibody binding to a biological target are well known in the field. Typically, antibody enrichment techniques employ protein-protein and/or protein-peptide, protein-oligosaccharide and potentially protein-nucleic acid interactions. All suitable means of antibody enrichment are contemplated. Suitably, means of antibody enrichment may generally be by antigen display techniques (phage, yeast, cis), immunisation (natural species, transgenic species expressing antibodies of other species, such as humans, e.g. Omnimouse, or VHH animals such as Llama, Alpaca), cell-free antibody selection, and/or library techniques (synthetic libraries, naïve libraries, VHH libraries, single chain libraries).

In embodiments, the means of antibody enrichment is via a technique such as phage display. In some embodiments, antibodies may be prepared by administering an immunogen to an animal or a transgenic animal that has been modified to produce intact human antibodies. In some embodiments, antibodies are made by hybridoma-based methods or by screening combinatorial libraries (e.g. yeast display, cis-display etc.) followed by panning or antibody screening with a synthetic scFv phage library obtained by design and/or from commercial sources, against the target, either as bound recombinant target (e.g. protein or peptide) or expressed on the surface or a suitable cell line, such as HeLa cells.

In embodiments that make use of the target protein expressed on the surface of cells, the cell lines required for panning may be transfected, transduced or electroporated using standard methods to achieve surface expression of the target protein. In embodiments, transfected cells may be selected for stable plasmid integration, single cell sorted and/or surface expression quantified, for example by standard methods such as by flow cytometry.

In embodiments, the synthetic phage library is then panned against either a recombinant target such as a protein or ligand or antigen and/or cells expressing the target on their surface and/or biological samples expressing the target protein (tumor/tissue biopsies for example).

In embodiments that rely on panning against a recombinant target such as a protein or antigen, screening plates may be coated with the target in a buffer over a suitable time period, for example, overnight.

In embodiments that rely on panning against a target such as a protein or antigen expressing cells, the cells are suitably blocked with a blocking buffer prior to the screen to improve specificity.

In embodiments that rely on panning against targets such as a protein or antigen expressing cells, non-specific binding phage particles are negatively selected by pre-incubation of the phage particles with blocking buffer coated wells or the selected cells, lacking target protein expression.

In some embodiments, where the target protein is present in the cell, the cell may be modified by genome editing or siRNA or shRNA or miRNA-based shRNA technology to remove or reduce target protein or antigen expression in the cell in order to allow for negative selection by pre-incubation of the phage particles.

For scFv selection, in embodiments, phage particles are added to the wells coated with adherent recombinant target protein or containing target protein or ligand or antigen expressing cells and incubated for a suitable length of time, for example 1 hour. After incubation, unbound and non-specifically bound phages are washed away by rinsing the wells. Bound phages, those that have affinity with the target, are eluted, and the eluate collected and neutralised as necessary. The eluate can then be used to infect exponentially growing E. coli TG1 cells. TG1 cells and phage eluate are mixed and e.g. incubate for 45 min at 37 C and 250 rpm, to allow for the infection of the TG1 cells by the virions. The panning may be repeated for additional cycles to enrich for the phage particles expressing the highest affinity binding.

In embodiments, after each, or selected, cycles of phage panning bound virions are eluted and further amplified for the next round. At the same time, in embodiments, the number of eluted virions may be counted and single clones are either sequenced or assayed or both. In embodiments, PCR can be performed with primers specific for the scFv or single domain chain flanking regions used in the phage library. Suitably, PCR can be cleaned up and next-generation amplicon sequencing performed according to standard procedures, for example, by ligation of adapters, followed by sequencing on a MiSeg™ NGS sequencer (Genewiz™ NGS sequencing).

In embodiments that rely on immunization with a target such as a protein, peptide or vector (e.g. RNA, AAV, plasmid) encoding for the target protein or peptide, animals are immunized following standard procedures, e.g. one primary injection and two booster immunizations. Immune response is monitored by tail bleeding the animal and assessing serum samples in an ELISA against the antigen. Spleen and/or lymph nodes and/or blood are harvested and cDNA is prepared for amplification of VH and VL sequences. Alternatively, target-specific B cells or bulk B cells can be sorted and light and heavy antibody chains can be analyzed via single cell sequencing technologies.

In embodiments that rely on VHH recognition domains, generated through immunization with a target such as a protein, peptide or vector (e.g. RNA, AAV, plasmid) encoding for the target protein or peptide, alpacas or llamas are immunized following standard procedures. After immunization immune response can be monitored by drawing blood. Lymphocytes are purified from blood and RNA is isolated from bulk lymphocytes, bulk B cells, or antigen-specific B cells. Single domain antibodies are PCR amplified from cDNA.

In embodiments that rely on design and/or prediction and/or optimization of binding domains through computational tools and/or machine learning methods, the resulting binding domains (scFv, VHH, receptor-based, anticalin) can be synthesised through DNA synthesis.

In embodiments, computational tools and/or machine learning methods may also be used for the design and/or prediction and/or optimization of any part of the CAR (for example, hinge region, transmembrane region, intracellular signalling region), or may be applied to the complete CAR structure to design and/or predict and/or optimize a part of the CAR, or the CAR as a whole. The resulting CARs can be synthetized through DNA synthesis.

CAR Cloning

Sequence Amplification and Ligation into CAR Scaffold

To generate the CAR library the identified or desired recognition sequence must be incorporated into a full CAR construct prior to introduction into an appropriate cell or cell line. Any suitable method for incorporating a recognition sequence into a CAR construct is contemplated.

In embodiments, desired recognition sequences, suitably scFv encoding sequences may be PCR amplified from the enriched phage display library or equivalent. Resulting PCR product may be digested and ligated at a suitable ratio into a CAR construct, for example a CAR vector, for example a lentiviral CAR scaffold vector. In some embodiments, the vector is a retroviral vector, a DNA vector, an RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

In embodiments, the CAR construct, suitably as a vector, encodes a number of domains, including but not limited to one or more of a promoter, a signalling peptide, one or more tags, a multiple cloning site containing restriction sites, a hinge; a transmembrane sequence, a co-stimulatory domain and a signalling domain. Alternatively, a suitable costimulatory domain is used. Any part of the desired recognition sequence or domain or component of a CAR scaffold may be seen as a potential point of variability in the putative CAR library, and subsequently produced, CAR-cell library, where the CAR library has been transduced into a suitable cell or cell line. In embodiments, any component part of the CAR or CAR vector (i.e. one or more of a promoter, a signalling peptide, one or more tags, a multiple cloning site containing restriction sites, a hinge; a transmembrane sequence, a co-stimulatory domain and a signalling domain), may be incorporated into the CAR construct from an individual sequence or a library of sub-sequences, prepared for combination with the other components of the CAR prepared as described above for the recognition sequence or by any other known manner.

In embodiments, desired recognition sequences, suitably scFv encoding sequences, may be PCR amplified from the enriched phage display library or equivalent. Resulting PCR product may be assembled by joining multiple fragments of the CAR in a single molecular cloning reaction such as Gibson assembly, restriction enzyme ligation, gateway cloning, golden gate assembly. CAR fragments can be a backbone, a multitude of promoters, a multitude of extracellular domains, a multitude of transmembrane domains and/or a multitude of intracellular domains.

In embodiments, the promoter is a human EF1a promoter, alternatively a human PGK promoter, SFFV promoter, truncated EF1a promoter, human CMV promoter, murine CMV promoter, murine EF1a promoter, UBC promoter, CAG promoter or LTR promoter. In embodiments, the signalling peptide is a CD8a signalling peptide, or a GM-CSF or TCR or a IgGI heavy chain derived signaling peptide. In embodiments, optional the tag present or absent and the tag is a Strep-Tag, a Myc-Tag, a HA-tag, a HiBiT-Tag, a Flag-Tag, a His-Tag. In embodiments, the multiple cloning site containing restriction sites comprises XbaI and SpeI restriction sites, EcoRI, XhoU, Kpn2I, BamHI, BbsI, Esp3I or other restriction sites or Gibson assembly is used with overhanging homology arms.

In embodiments, the extracellular domain comprises a hinge region (or spacer/stalk region). In another embodiment, the extracellular domain is derived from (e.g. comprises) CD8a, CD28, CD28T, OX40, 4-1BB/CD137, CD2, CD3, CD4, CD5, CD8, CD9, CD16, CD22, CD37, CD40, CD45, CD80, CD86, PD1, NKG2D, TNFR2, IgG, IgA, IgM, IgD, IgE, The extracellular domain can be derived from a natural or synthetic source.

In embodiments, the transmembrane domain can be designed to be fused to the extracellular domain in the costimulatory domain. In one embodiment, the transmembrane is used that is naturally associated with the extracellular domain or the intracellular costimulatory domain. In other embodiments, the transmembrane domain can be selected from natural or synthetic source. Where the source is natural, the transmembrane domain can be derived from any membrane-bound or transmembrane protein. In some embodiments, the transmembrane domain is derived from CD28, CD137, CD8a, CD3, CD2, CD4, CD5, CD7, CD8, CD9, CD16, CD22, CD37, CD40, CD45, CD80, CD86, CD278 PD1, NKG2D, TNFR2. In embodiments, the chimeric receptor contains a co-stimulatory domain. Costimulatory molecules include, but are not limited to an MHC class I molecule, TNF receptor proteins, Immunoglobulin-like proteins, cytokine receptors, integrins, signalling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-166 (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11 b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

The intracellular signalling domain can comprise a ITAM (immunoreceptor tyrosine-based activation motif) signalling motif. In embodiments, the signalling domain can be derived from CD247 (CD3z), FcR gamma, common FcR gamma (FCER1G), Fc gamma RIIa, FcR beta (Fc Epsilon Rib), CD3 gamma, CD3 delta, CD3 epsilon, CD22, CD79a, CD79b, CD278 (“ICOS”), FcERI, CD66d, CD32, DAP10, and DAP12 signalling domain.

Alternative CAR Synthesis

In an alternative embodiment for cloning CARs in accordance with the present invention, sequencing, for example, NGS sequencing or single cell sequencing of the enriched scFv sequences or antibodies or VHHs via amplicon sequencing, is used to provide the target-enriched recognition sequence. The most relevant antibodies, VHH or scFvs may then be converted in a scFv format, if required, synthesized and cloned into a CAR construct, suitably as a vector although any suitable technique is envisaged. Alternatively, an antibody sub-library of 50-10⁷ CARs with the enriched recognition sequences may be synthesized and cloned into a CAR construct. Alternatively, a DNA or RNA fragment can be synthetized, encoding the CAR sequence.

In embodiments, and in accordance with methods known in the art, the CAR sequence does not need to be cloned, e.g. an mRNA library of CARs that are electroporated or a CAR library that are inserted in the genome using genome editing.

Any combination of recognition sequence, or part thereof, and individual domains of a CAR may be combined to provide a single representative within a CAR library or the subsequent CAR-cell library prepared therefrom. To achieve high-throughput screening of CARs against a new or established target, the ability to combine different recognition sequences and the individual domains of a CAR construct in a straightforward and familiar manner provides a previously unmet need to achieve diversity in a CAR-cell library that can rapidly be prepared and the performance of an identifiable CAR structure may be evaluated in a clinically relevant context.

In certain embodiments, the invention is directed to a cell comprising the polynucleotide, the vector, the polypeptide, or any combination thereof encoding a CAR. Any vector known in the art can be suitable for the present invention. In some embodiments the vector can be stably integrated in the genome of the cell. Such methods can comprise retroviral vectors, lentiviral vectors, transposons (sleeping beauty, piggyBac) or genome editing mediated insertion of a desired sequence via homologous recombination, such as CRISPR/Cas9 mediated insertion. In some embodiments, the vector is not stably integrated and introduced as a vector via methods such as plasmid electroporation, mRNA electroporation, AAV delivery, non-integrating lentiviral vectors.

In some embodiments the cell is a T cell, a NK cell, a NK-T cell, a regulatory T cell, an iPSC cell, a gamma-delta T cell, a macrophage, a monocyte, a CD4+ T cell, a CD8+ T cell, or any cell enriched via specific cell surface markers via fluorescence activated cell sorting. In some embodiments the cell is derived from iPSC or other pluripotent cells. In some embodiments this cell is derived or isolated from human tissue/PBMCs, murine tissue, rat tissue, non-human primate tissue, hamster tissue, rat tissue. In some embodiments the cell is a cell line such as Jurkat, SupT1, ALL-SIL, HeLA, EL4,TK-1, CHO, HEK293T. In some embodiments, the cell has been modified with a reporter gene, such as NFAT-luciferase, IL2-luciferase, NF-kB-luciferase, NFAT-GFP, NF-kB-GFP, IL2-GFP.

In some embodiments, the cells comprising the polynucleotide may optionally be enriched using selection methods such as magnetic enrichment, fluorescence activated cell sorting (FACS) using markers co-expressed with the CAR. These markers can be but are not limited to GFP, BFP, mTAGBFP2, mCherry, truncated CD34, truncated CD19, truncated EGFR, truncated LNGFR Strep-tag, Twin-Strep-tag, HA-tag, His-Tag and can be linked to the CAR via an IRES, a 2A peptide or directly fused to the CAR. In some embodiments, the cells comprising the polynucleotide may optionally be selected using resistance genes such as puromycin, hygromycin B, blasticidin, Zeocin.

Library Sequencing

In embodiments, the resulting library of CARs may be sequenced, for example via NGS sequencing, to verify coverage of all enriched scFv sequences.

### CAR-Cell Library Screening

The invention, in a further aspect, also relates to improvements in the screening of CAR candidates when expressed in a cell. Any suitable method for high throughput screening of the CAR library is contemplated.

In embodiments, after the CAR-cell library generation, direct testing of CAR functionality of the library may suitably be by one of the following methods:

1. Individualised Parallel High Throughput CAR Processing

This protocol allows for the automation and high throughput screening of between 50 to 500 CARs in parallel. In embodiments, the testing is done initially in a format in which individual CARs are expressed in an individual cell in a given test environment, for example in an individual well of a multi-well plate. In embodiments, the cells are then subjected to one or more suitable high throughput screening assays, such as functional reporter assays, for example, ELISA to quantify cytokine production or cytotoxic activity. As the protocol requires CARs to be expressed in an individual cell, which can be identified by location in a plate or by suitable marking such as a physical barcode or QR code that can be read then rapid identification of a CAR of interest is possible.

2. Pooled or Mixed CAR Library Screening with Subsequent Deconvolution by Sorting/Enrichment

This protocol processes a CAR library as single, pooled, or mixed samples of all generated CARs present on one or more cells. Suitably, the CAR library is tested or screened via positive enrichments using suitable target antigens or ligands. In embodiments, the CAR is activated via a ligand and the activated cells can be identified and sorted/enriched through any suitable means known to the skilled person. In embodiments, the identification of activated cells may be based on the up-regulation of activation markers (for example, CD69, 41BB, OX40, CD25 etc.), and/or cytokines (e.g. IFN gamma, IL2, TNF alpha) and/or introduced reporters (e.g. NFAT, NfkB response elements driving expression of a reporter gene, such as eGFP, mCherry, BFP, tCD34, tCD19) and FACS sorting or magnetic enrichment. In embodiments, the identification of unspecifically activated cells or tonic signaling CARs may be based on the up-regulation of activation markers, and/or cytokines and/or reporters in the absence of the ligand and FACS sorting or magnetic enrichment. The sequence of CARs expressed in the calls can be identified via sequencing, for example next generation sequencing.

The application of next generation sequencing methods allows for the rapid identification of the sequence of the CAR or part thereof in the CAR cell of interest. This rapid deconvolution of the high-throughput screen output to the point where the sequence, or part thereof, of the CAR of interest is known, is a particularly important aspect of the present invention.

3. CAR Library Screening Via Single Cell RNA Sequencing

The rapid analysis of a high-throughput screen of a CAR library, including rapid identification of the sequence of the CAR following identification of a CAR cell of interest can be increased further through the use of single cell RNA sequencing techniques. This also allows for identification of more than one CAR present in a cell, when single, pooled or mixed CAR cell libraries are used.

This protocol processes a CAR library as single, pooled or mixed samples of all generated CARs present on one or more cells. This protocol introduces individual CARs into a cell (e.g. primary human T cell) to provide a discrete CAR-cell library. The cells are then exposed to the ligand or antigen as for screening method (2) above. In this case, however, samples will be taken for non-activated cells (ligand absent) and activated cells (ligand present). RNA single cell sequencing libraries can then be prepared as samples from the initial CAR-cell library and cells assessed for differential expression between non-activated and activated states. This method allows to assess change in the expression of any gene normally assessed in traditional screening methods (cytokine production, toxicity mediators, T cell exhaustion, naive/memory phenotype of the cell etc.). The information of which cell expresses which CAR is then linked to the RNA sequencing data so those CARs that resulted in activation of the cell can be rapidly identified. In embodiments, the method of linking the RNA sequence data to the CAR responsible uses long-read sequencing to link the barcode information of the library to the CAR sequence, or part thereof.

In embodiments, none of the above methods require pre-selection of the recognition sequences, using phage display or immunization, for example. Method 1 may make use of pre-selection to generate the recognition sequence library thereby breaking the selection of candidate CARs into a two-stage process, whereas method 2 and method 3 can be performed without any pre-selection, although pre-selection in some form in these methods is not excluded.

### Rapid Iterative Optimisation of the Screening Results

Rapid or high-throughput identification of sequence information of the CARs of interest, or parts thereof, and linking this to functional properties obtained for the CAR-cell from which it originates is an important aspect of the present invention. This allows for large amounts of data to be obtained which can show trends and cluster information that would otherwise be impractical to achieve, for example, using prior art genome isolation and PCR sequencing methods of individual samples. This expansive, information-rich, rapid high-throughput analysis allows for rapid cycling and iteration of the CAR sequence to optimise a given result.

As an example, sequence information obtained from screening and single cell RNA sequencing described above, can be analysed and different gene signatures defined. Machine learning methods can then be used to define functional clusters and signatures such as tonic CAR activation in the absence of target cell.

Furthermore, with such a large amount of data available linking structure of the CAR to function then further trends can be identified, such as a preference for a particular structural domain (hinge domain, transmembrane domain, or intracellular domain or combination thereof).

When the high-throughput screen is directed more at identifying novel CARs or part thereof for a given receptor, such rapid linking of sequence data to function of the CAR can lead to rapid identification, and optionally, optimisation of novel CAR structures, in particular, identification, and optionally, optimisation of novel antigen binding fragments, based on CAR function alone, rather than antibody function as used to date.

### Machine Learning-Assisted Analysis

With the large amounts of data made available by the rapid, high-throughput analysis methods described above, machine learning may be employed to analyse the data and define criteria of interest to assist in selection of CARs of interest. CARs with high clinical overall response rate can then be identified. CAR sequences can then be ranked and new sequences of interest identified.

It is to be appreciated that a high-throughput screen of a CAR-cell library, as defined herein, must be considered to encompass (1) high-throughput preparation of the CAR-cell library in an efficient and accurate manner, (2) functional testing of the members of the CAR-cell library in an efficient and accurate manner, and (3) analysis of the results to link the sequence of the CAR in each member of interest in the CAR-cell library, as defined by a desire function. Optionally, step (4) to interpret the data from the screen to rapidly optimise the result to improve the functional outcome, all based on the primary function of the CAR, which is the ultimate therapeutic is also an important step.

Each of the above screening methods described above is discussed in more detail below.

a) Individualised Parallel High Throughput CAR Processing

Selection of Clones

In embodiments, high-throughput screening of CARs may be performed in an individual, parallel format, for example, with 48, 96, 192, 288, 384, 480, 576 or more different CARs. In order to parallel screen multiple CARs, a recognition sequence library may be prepared as described hereinabove, or otherwise, to afford a degree of pre-selection, or may be obtained or synthesised without pre-selection, by any suitable means, or obtained from a commercial source.

In embodiments, the recognition sequences of the recognition sequence library, typically without any need for evaluation or sequencing, are then cloned as a CAR library that encode for the complete CAR, i.e. comprising a recognition sequence or scFv, a hinge domain, a transmembrane domain, and an intracellular domain. The members of the CAR library may be cloned as plasmids or in another suitable form to enable expression of the CAR in a cell. In such embodiments the CAR sequence may also include any required ancillary functional components such as promoter, expression or reporter sequences.

In embodiments, the members of the CAR library, suitably as plasmids are then isolated. In embodiments, the resulting members of the CAR library/plasmids may be tested for the recognition sequence via Sanger sequencing or via NGS sequencing.

Transduction/Transfection/Electroporation of Clones into Cells

In embodiments, the individual members of the CAR library are then transfected into suitable individual cells to provide a CAR-cell library where each member cell expresses an individual CAR. In embodiments where the CAR library is present as plasmids that are to be used to produce viral particles, the plasmids are co-transfected with packaging plasmids into a suitable cell line. In these embodiments, the resulting viral vectors are then harvested at a suitable time after transfection.

In embodiments, the plasmids or viral vectors are used to transduce a suitable cell line, for example a Jurkat cell line expressing a suitable reporter construct, such as an NFAT-luc2 reporter construct, or primary human T cells. In embodiments, cells are selected in order to enrich for transduced cells, for example through use of puromycin, where a puromycin resistance gene was co-expressed with the CAR.

In alternative embodiments, the plasmid, DNA or RNA encoding the CAR are electroporated, for example by, nucleofection into suitable cells.

### Testing of Cells

For testing, the transduced cells may be co-cultured with target-expressing cells, such as HeLa cells or Rpmi 8226 cell line. At a suitable time thereafter, for example 24 hours, the activity of a reporter of CAR function can be measured in the CAR expressing cells. Suitably, luciferase reporter activity may be measured in the CAR expressing cells in order to assess CAR downstream activity.

In embodiments that make use of transduced T cells, these may be co-cultured with target-expressing cells such as HeLa or Rpmi 8226 cell lines, constitutively expressing a suitable reporter, such as a luciferase reporter. Killing was assessed by measuring reporter, suitably luciferase, intensity relative to a control CAR construct. In embodiments, supernatants may be used to determine other measures of CAR activity such as IFNg and IL2 production that are produced by CAR-T cells after activation.

In this way, activity of the CAR may be measured in a high-throughput manner and compared to results for other CARs in the library, thereby allowing selection of the most promising CARs based on their function in response to the target or antigen.

b) Pooled or Mixed CAR Library Screening with Subsequent Deconvolution by Sorting/Enrichment

Recognition Sequence Library and CAR Scaffold Preparation

In this protocol, a recognition sequence library, suitably a pooled recognition sequence library, may be prepared as described hereinabove, or by any suitable means, or synthesised or obtained from a commercial source.

In embodiments, the prepared sequence library may be sequenced by suitable means, such as next-generation sequencing.

CAR Scaffold Preparation

The remaining components that are required for a functional CAR (including but not limited to, a hinge domain, a transmembrane domain, an intracellular domain and optionally a co-stimulatory domain) are prepared as a CAR scaffold. Each individual component may be represented by a group of one or more members. The CAR scaffold may be prepared by combination of each member of each group of individual components. The combination may be via combinatorial or pooling methods or by directed and/or selective combination of individual members of each group. In embodiments, the CAR scaffold is pre-selected to contain a single member from each group of components. In alternative embodiments, a CAR scaffold library is generated of multiple varied CAR scaffolds.

CAR Library Cloning and Pooled Transduction/Transfection/Electroporation

In embodiments, the recognition sequence library was subsequently used for CAR library preparation where the recognition sequence is cloned with the CAR scaffold or each CAR scaffold within a CAR scaffold library. Suitably each member of the CAR library is provided as a vector, suitably a lentiviral vector although any means as herein described or otherwise known in the art may be used to prepare a CAR library of cells encoding CARs with the desired set of recognition sequences.

It is to be noted that while the method of generating the CAR library above requires the combination of a recognition sequence library with a CAR scaffold or CAR scaffold library, wherein each CAR scaffold comprises a hinge domain, a transmembrane domain, an intracellular domain and optionally a co-stimulatory domain, it is contemplated that any method of generating a CAR library is contemplated. For example, a CAR library may alternatively be formed by cloning libraries of each individual component of a CAR required for functionality, i.e. libraries comprising one or more members of: a recognition sequence; a hinge domain, a transmembrane domain, an intracellular domain and optionally a co-stimulatory domain may be prepared and suitably combined to form a CAR library. Such combination may be via combinatorial or pooling methods or by directed and/or selective combination of individual members of each group. Suitably such CAR libraries may be cloned as vectors, suitably as lentiviral vectors as above.

In embodiments where the members of the CAR library are provided as vectors, suitable cells, such as HEK293T cells were seeded, transfected with the recognition sequence library, optionally with suitable packaging plasmids, such as 3rd generation lentiviral packaging plasmids by FuGene-based transfection to provide the required CAR library.

The viral vectors of the CAR library thus produced were prepared, typically involving adjusting the concentration, and titration before the library is optionally sequence verified by suitable sequencing, such as next-generation sequencing.

A suitable cell such as Jurkat or T-cells, e.g. primary human T cells were transduced with the CAR library as vectors encoding the members of the CAR library and any suitable controls to provide mixed or pooled or mixed samples of cells, each cell comprising an individual CAR from the CAR library.

In embodiments, T-cells, for example, primary human T cells may be isolated by immunoaffinity-based enrichment. In embodiments, this enrichment may make use of leukapheresis samples from human donor subjects. In embodiments, T-cells are isolated from leukapheresis samples from human donor subjects. T cells were activated, for example, with TransAct™ (Miltenyi Biotech™) in the presence of IL-2 (Miltenyi Biotech™) and/or IL-7 and/or IL-15 and transduced with the CAR vectors of the CAR library to provide a CAR-cell library.

The viral vectors thus produced were prepared, typically involving concentration, and titration before the library was sequence verified by suitable sequencing, such as next-generation sequencing.

In some embodiments, the plasmid, DNA or RNA encoding the CAR library are transfected or nucleofected or electroporated into suitable cells.

Optional Selection of Transduced Cells

After transduction and expansion, in embodiments, transduced cells may be selected. In embodiments where a puromycin resistant gene was co-expressed with the CAR, this may be achieved by exposure to puromycin for a suitable period of time, for example 2 days.

In embodiments, after transduction and expansion, transduced cells may be selected/enriched by any suitable means. In embodiments, the cells comprising the polynucleotide may optionally be enriched using selection methods such as magnetic enrichment, fluorescence activated cell sorting (FACS) using markers co-expressed with the CAR. These markers can be but are not limited to GFP, BFP, mTAGBFP2, mCherry, truncated CD34, truncated CD19, truncated EGFR, truncated LNGFR Strep-tag, Twin-Strep-tag, HA-tag, His-Tag and can be linked to the CAR via an IRES, a 2A peptide or directly fused to the CAR. In some embodiments, the cells comprising the polynucleotide may optionally be selected using resistance genes such as puromycin, hygromycin B, blasticidin, Zeocin. Suitably, where a truncated CD34 marker is co-expressed with the CAR, this may be achieved with anti-CD34 microbeads (Miltenyi Biotech™).

Sampling of Baseline/Non-Activated Cells

In embodiments, in order to deplete CARs with undesired high background activation and tonic signalling, a first selection step may be performed. In embodiments, this comprises depleting cells positive for activation markers by known methods, for example for the activation marker CD69, anti-CD69 biotin antibody and anti-biotin magnetic beads (Miltenyi Biotech™) may be used. In embodiments, the activation marker may be IFN gamma, CD25, OX40. In embodiments, an engineered cell is used where a marker is inserted in the genome under the control of an endogenous activation responsive promoter, such as IL2, IFN gamma, CD69. In embodiments, an engineered cell is used where the promoter is a synthetic promoter, such as NFAT, Nf-kB and controlling the expression of a reporter such as GFP, BFP, mCherry, tCD34, tCD19, lunciferase. In embodiments enrichment is achieved using magnetic enrichment or FACS. In embodiments, genomic DNA of the negative fraction was isolated, and the scFv sequences are PCR amplified and sequenced by NGS sequencing.

Sampling of Cells after Activation (NFAT/NfkB, CD69, IFNg, IL2, Reporter Constructs)

Cells, or in embodiments where the first selection step above has been performed, activation marker-depleted or otherwise selected cells, were cultured in the presence of target-expressing cells, such as HeLa or Rpmi 8226 cells or K562 cells. Cells were then selected based on CAR function. In embodiments CAR function may be assessed by any suitable means. In embodiments, CAR function can be measured as i) CD69 up-regulation via CD69 biotin and anti-biotin magnetic beads (Miltenyi Biotech™) or ii) IFNg production by selection with the IFN-γ Secretion Assay—Cell Enrichment and Detection Kit (Miltenyi™) iii) fluorescence activated cell sorting of IL-2 GFP or NFAT-GFP or Nf-kB-GFP up-regulating cells. Genomic DNA of positive clones was isolated by standard means, ScFv sequences were PCR amplified and enrichment was assessed via suitable sequencing techniques.

c) CAR Library Screening Via Single Cell RNA Sequencing

CAR Library Cloning and Preparation

In this protocol, a recognition sequence library, suitably a pooled or mixed scFv library, may be prepared as described hereinabove, or by any suitable means, or synthesised or obtained from a commercial source.

In embodiments, the prepared recognition sequence library may be sequenced by suitable means, such as next-generation sequencing.

CAR Scaffold Preparation

A CAR scaffold or CAR scaffold library may be prepared as described hereinabove.

Pooled Transduction/Transfection/Electroporation

In embodiments, the recognition sequence library was subsequently used for CAR library preparation, where the recognition sequence is cloned with a CAR scaffold, suitably as a vector, suitably a lentiviral vector, although any means as herein described or otherwise known in the art may be used to prepare a CAR library of cells encoding CARs with the desired set of recognition sequences.

As above, it is to be noted that while the method of generating the CAR library above requires the combination of a recognition sequence library with a CAR scaffold or CAR scaffold library, wherein each CAR scaffold comprises a hinge domain, a transmembrane domain, an intracellular domain and optionally a co-stimulatory domain, it is contemplated that any method of generating a CAR library is valid. For example, a CAR library may alternatively be formed by cloning libraries of each individual component of a CAR required for functionality, i.e. libraries comprising one or more members of: a recognition sequence; a hinge domain, a transmembrane domain, an intracellular domain and optionally a co-stimulatory domain may be prepared and suitably combined to form a CAR library. Such combination may be via combinatorial or pooling methods or by directed and/or selective combination of individual members of each group. Suitably such CAR libraries may be cloned as vectors, suitably as lentiviral vectors as above.

In embodiments where the members of the CAR library are provided as vectors, suitable cells, such as HEK293T cells were seeded, transfected with the scFv library, optionally with suitable packaging plasmids, such as 3rd generation lentiviral packaging plasmids by FuGene-based transfection to provide the required CAR library.

The viral vectors of the CAR library thus produced were prepared, typically involving concentration, and titration before the library was sequence verified by suitable sequencing, such as next-generation sequencing.

A suitable cell line such as Jurkat or T-cells, e.g. primary human T cells were transduced with the CAR vectors encoding the members of the CAR library and any suitable controls to provide individual or mixed or pooled or mixed samples of cells. In embodiments, T-cells, for example, primary human T cells may be isolated by immunoaffinity-based enrichment. In embodiments, this enrichment may make use of leukapheresis samples from human donor subjects. In embodiments, T-cells are isolated from leukapheresis samples from human donor subjects. T cells were activated, for example, with TransAct™ (Miltenyi Biotech™) in the presence of IL-2 (Miltenyi Biotech™) and/or IL-7 and/or IL-15 and transduced with the CAR vectors of the CAR library to provide a CAR-cell library.

In some embodiments, the plasmid, DNA or RNA encoding the CAR library are transfected or nucleofected or electroporated into suitable cells.

Optional Selection of Transduced Cells

In embodiments, after transduction and expansion, transduced cells of the CAR-cell library may be selected/enriched by any suitable means. In embodiments, the cells comprising the polynucleotide may optionally be enriched using selection methods such as magnetic enrichment, fluorescence activated cell sorting (FACS) using markers co-expressed with the CAR. These markers can be but are not limited to GFP, BFP, mTAGBFP2, mCherry, truncated CD34, truncated CD19, truncated EGFR, truncated LNGFR Strep-tag, Twin-Strep-tag, HA-tag, His-Tag and can be linked to the CAR via an IRES, a 2A peptide or directly fused to the CAR. In some embodiments, the cells comprising the polynucleotide may optionally be selected using resistance genes such as puromycin, hygromycin B, blasticidin, Zeocin. Suitably, where a truncated CD34 marker is co-expressed with the CAR, this may be achieved with anti-CD34 microbeads (Miltenyi Biotech™).

T Cell Activation

A CAR-cell library, in embodiments, formed of primary human T-cells expressing the desired CARs are activated by the target. In embodiments, the cells expressing the desired CARs are co-cultured with, in embodiments, adherent cells, such as HeLa cells, expressing the target, such as target protein, ligand or antigen. Alternatively, the CAR cell library may be activated with the target, for example, a recombinant protein target, adherent on a suitable surface, such as a multi well plate or bound to beads. In some embodiments the CAR-cell library may be exposed to control cells lacking the target antigen or plates coated with a control protein/peptide or blocking buffer. After activation, cells from the supernatant are harvested in order to avoid contamination with adherent cells. In some embodiments the T cells can be positively selected to remove contaminating cells or the contaminating cells can be negatively selected/depleted using magnetic beads or FACS.

Single Cell RNA Sequencing

RNA libraries of harvested single-cell suspensions are prepared using standard means (see example). In embodiments, samples were quality controlled to have sufficient numbers of genes detected, a high percentage of reads mapped to the respective genome, and sufficient number of cells detected. CAR activation can be assessed by comparing expressions of transcripts between non-activated and activated cells, expressing the same CAR. In some embodiments, transcripts that can help identify activated cells are, but not limited to any single or combination of CD69, IL2RA, IFNG, CCL4, CCL3, TNFRSF4, TNF, CD3E, CD4, CD28, CD83, TNFS14, JUNB, MYC, FOSL2.

Linking of scFv/Recognition Sequence to Cell Barcode by Long Read Sequencing

The single cell RNA sequence information is then linked to the structure and sequences of the CAR library. Any suitable means for doing this is contemplated. In one exemplary embodiment this is achieved by performing long-read sequencing under standard techniques. To this end, the relevant scFv/recognition sequence of the single cell library, with each member suitably identified, for example by a barcode or QR code, was PCR amplified. Optionally, a second nested PCR may be performed to further enrich for specific PCR products using standard techniques. PCR products were purified and sequenced, for example by preparation with the Ligation Sequencing Kit (Oxford Nanopore) and sequenced on a Minion™ sequencing device according to manufacturers instructions.

Each of the above methods of screening allow for rapid and robust testing of CAR function in a clinically relevant setting. Once prepared the CAR-cell library may be subjected to each or any of the screening methods above under conditions to test any suitable output. The pooled or mixed methods (multiple CARs per cell, or multiple single CAR cells per well) offer particular advantages in speed of testing. Robust and accurate methods of deconvolution of individuals CAR structures from pooled hits is provided. In this way, an efficient and rapid method of high-throughput identification of potential CAR clinical candidates is provided.

The invention is further illustrated by the following non-limiting example.

In a specific embodiment, the process of generating a CAR-cell library and selecting a CAR clinical candidate comprises one or more of the following steps:

1) Selection of Biological Target

A target is selected based on appropriate knowledge and understanding from the literature, databases, etc. The target may be any suitable biological entity that may be recognised by an antibody, or an scFv of a CAR. Suitably, the target can be selected from the group consisting of: proteins; peptides, MHC presented peptides, MHC-like presented peptides. The target chosen for this example is the protein B-cell maturation antigen (BCMA).

2) Antibody Enrichment.

Cell Line Preparation or Recombinant Protein

Recombinant human (h)BCMA-Fc fusion protein (catalog #193-BC-050) and recombinant mouse (m)BCMA-Fc fusion protein (catalog #593-BC-050) were obtained from R&D Systems™.

Vectors expressing human BCMA and mouse BCMA were transiently transfected into HeLa cells using standard methods. Transfected HeLa adherent cells were selected for stable plasmid integration using zeocin, then single cell sorted and BCMA surface expression was quantified by Flow Cytometry using anti-human BCMA-PE antibody (R&D Systems™; FAB 193P).

Phage Panning/Mammalian Display

A synthetic human scFv phage library was panned against recombinant human BCMA protein and HeLa cells expressing human BCMA. The library was grown to log phase, and then rescued with M13KO7 helper phage (Antibody Design Lab, PH010L) before being amplified overnight at 25° C. in a shaker. The phage library was subsequently precipitated with PEG/NaCl, re-suspended in PBS and stored at −80° C. For panning against recombinant hBCMA, microplates or Protein G coated magnetic beads were coated with hBCMA protein in PBS at 4° C. For panning with BCMA expressing HeLa cells, the cells were blocked for 1 h with a blocking buffer at room temperature. For pre-selection, phage particles were pre-incubated with the blocking buffer and Fc control protein in microplate wells or Protein G coated magnetic beads or wild-type HeLa cells or HeLa cells expressing a control protein. After pre-incubation, phage particles were added to the wells coated with hBCMA or hBCMA coated magnetic beads or BCMA-expressing HeLa cells and incubated for 1 h. After incubation, unbound and non-specifically bound phages were washed away by rinsing the wells with PBCT. Bound phages were eluted by 100 mM triethylamine (TEA), and the eluate was neutralized by 1 M Tris-HCl (pH 7.4). The eluate was then used to infect exponentially growing E. coli TGI cells. The panning was repeated for an additional two cycles.

NGS Sequencing of Antibodies/scFvs/VH Only

After each cycle of phage panning a sample was used to amplify the enriched scFv sequences. PCR was performed with primers specific for the scFv flanking regions. PCR was cleaned up and next-generation amplicon sequencing was performed according to standard procedures. In brief, the library was prepared by ligation of adapters, followed by sequencing on a MiSeg™ NGS sequencer (Genewiz™ NGS sequencing). Alternatively, PCR amplicons were barcoded with PCR barcoding kit or Native Barcoding kit (Oxford Nanopore Technologies™) and sequenced on a MinION™ sequencing device (Oxford Nanopore Technologies™).

3) CAR Cloning into Backbone

PCR Amplification and Ligation into CAR Scaffold

ScFv encoding sequences were PCR amplified from the enriched antibody library using NEB Q5 2× master mix (M0492L). Resulting PCR product was digested with XbaI and SpeI (both NEB) and ligated at a 3:1 ratio into a pre-digested lentiviral CAR scaffold vector using T4 DNA ligase (NEB, M0202L). The scaffold vector encodes a human EF1a promoter, followed by a CD8a signalling peptide, a Strep-Tag, a multiple cloning site containing XbaI and SpeI restriction sites, a CD8a stalk and transmembrane sequence, followed by a CD28 co-stimulatory domain and a CD247 (CD3z) signalling domain. Alternatively, a 4-1BB costimulatory domain was used. In another version, a CD28 stalk and transmembrane domain was used. Alternatively, the scFvs were ligated into a mix of CAR scaffolds differing in their hinge and transmembrane domain. Additionally, the plasmids contained a selectable truncated CD34 (tCD34) marker or a puromycin resistant gene, linked to the CAR by an IRES or 2A peptide sequence.

Subsequently, ligation was cleaned up by ethanol/sodium acetate DNA precipitation and electroporated into electrocompetent cells (Lucigen Endura™ cells (cat #60242)).

Alternative: CAR Plasmid Synthesis

Alternatively, NGS sequencing of the enriched scFv sequences via amplicon sequencing revealed the BCMA-enriched antibodies. Top 50 antibodies were synthetized by TWIST bioscience™ or Integrated DNA technologies™ (IDT™) and cloned into a CAR scaffold vector. Alternatively, an antibody sub-library of 10³-10⁷ CARs with the enriched scFvs was synthesized by TWIST bioscience™ and cloned into a CAR scaffold.

Library Sequencing

The resulting library of CARs was sequenced via NGS sequencing to verify coverage of all enriched scFv sequences.

4) CAR Screening

a) Individualised Parallel High Throughput CAR Processing

Selection of Clones and Mini Prep

High-throughput screening of CARs was successfully performed with 48, 96, 192, 288, 384, 480 or 576 different constructs. In order to parallel screen multiple CARs, single bacteria colonies were picked and grown overnight in deep-well 96-well plates. Plasmid isolation was performed using the Promega Wizard™ MagneSil™ Plasmid Purification System (A1630). Alternatively, the process was automated using a liquid handler (OT-2 Opentrons). Resulting plasmids were tested for the scFv sequence via Sanger sequencing or via NGS amplicon sequencing on a MiSeq machine (Genewiz™)

Transduction/Transfection/Electroporation of Clones into Cells

Plasmids were used to produce lentiviral particles in a 96-well format. To this end, the plasmids were co-transfected with 3rd generation packaging plasmids into HEK293T cells using FuGeneHD (Promega). The next day supernatant was exchanged and viral vectors were harvested on day 2 after transfection.

Viral vectors were used to transduce Jurkat cell line, expressing an NFAT-luc2 reporter construct, or primary activated human T cells. Cells were selected with puromycin in order to enrich for transduced cells.

Testing of Cells (NFAT, CD69, CD25, Toxicity, Potency, Cytokine . . . )

Subsequent to selection of transduced Jurkat cells, cells were co-cultured with BCMA-expressing HeLa cells or Rpmi 8226 cell line at a 1:1 ratio. 8 hours later luciferase activity was measured in Jurkat cells in order to assess CAR downstream activity. Similarly, transduced T cells were co-cultured with BCMA-expressing HeLa or Rpmi 8226 cell lines, constitutively expressing a luciferase reporter. Killing was assessed by measuring luciferase intensity relative to a control CAR construct. Supernatants were used to measure IFNg and IL2 production by CAR-T cells after activation.

b) Pooled or Mixed CAR Library Screening with Subsequent Deconvolution by Sorting/Enrichment

CAR Library Cloning and Preparation

Subsequent to the cloning of scFvs into the CAR backbone, electroporated bacteria were grown overnight on LB agar plates. The next day, colonies were harvested by adding 10 ml of LB medium to the plates and scraping the colonies off with a cell scraper. Liquid was transferred into a collection tube and pooled with the collected bacteria from the other LB plates. Collected bacteria were pelleted and plasmid maxi preparations (QIAGEN™) were performed. The prepared CAR library was sequenced by next-generation sequencing.

Pooled Transduction/Transfection/Electroporation

Plasmid library was subsequently used for lentiviral vector preparation. Here, HEK293T cells were seeded, transfected with the library and the 3rd generation lentiviral packaging plasmids by FuGene-based transfection. Viral vectors were concentrated, titrated and the library was sequence verified by next-generation sequencing.

Transduction of Cells and Optional Selection

Jurkat or primary human T cells were transduced with the lentiviral vectors encoding the anti-BCMA CAR library and controls. Primary human T cells were isolated by immunoaffinity-based enrichment from leukapheresis samples from human donor subjects. T cells were activated with TransAct™ (Miltenyi Biotech™) in the presence of IL-2 (Miltenyi Biotech™) and transduced with the lentiviral library at an MOI of 0.2. Jurkat cells were seeded at 2*10\hat{ }5 cells/ml and transduced at an MOI of 0.2.

After transduction and expansion, transduced cells were optionally selected with puromycin for two days, as a puromycin resistant gene was co-expressed with the CAR. Alternatively, transduced cells were selected with CD34-microbeads (Miltenyi Biotech™) on an LS column (Miltenyi Biotech™), when a tCD34 was co-expressed with the CAR.

Sampling of Binders, CAR-Expressers

In order to select CARs that are expressed on the cell surface and bind to the target of interest, CAR cell library can be stained with the target of interest, at a given concentration. CAR affinity/avidity determines if a given CAR will be binding at a given concentration. The target of interest can be conjugated to biotin, an Fc-tag fusion, a His-tag, a strep-tag or similar. The cells can be sorted by flow cytometry with a secondary antibody against the protein and/or the tag or fluorescence marker.

Sampling of Baseline/Non-Activated Cells

In order to deplete CARs with undesired high background activation and tonic signalling, a first selection step was performed, depleting CD69-positive cells, using anti-CD69 biotin antibody and anti-biotin magnetic beads (Miltenyi Biotech™). Genomic DNA of the negative fraction was isolated, and the scFv sequences were PCR amplified and sequenced by NGS sequencing.

Sampling of Cells after Activation (NFAT/Nfic13, CD69, IFNg, IL2, Reporter Constructs)

CD69-depleted cells were cultured in the presence of BCMA-expressing HeLa or Rpmi 8226 cells for 24 hours. Cells were then selected for i) CD69 up-regulation via CD69 biotin and anti-biotin magnetic beads (Miltenyi Biotech™) or ii) IFNg production by selection with the IFN-γ Secretion Assay—Cell Enrichment and Detection Kit (Miltenyi™). Genomic DNA of positive clones was isolated, scFv sequences were PCR amplified and enrichment was assessed via NGS.

c) CAR Library Screening Via Single Cell RNA Sequencing

CAR Library Cloning and Preparation

Subsequent to the cloning of scFvs into the CAR backbone, electroporated bacteria were grown overnight on LB agar plates. The next day, colonies were harvested by adding 10 ml of LB medium to the plates and scraping the colonies off with a cell scraper. Liquid was transferred into a collection tube and pooled with the collected bacteria from the other LB plates. Collected bacteria were pelleted and plasmid maxi preparations (QIAGEN™) were performed. The prepared CAR library was sequenced by next-generation sequencing.

Pooled Transduction/Transfection/Electroporation

The anti-BCMA CAR plasmid library was subsequently used for lentiviral vector preparation. Here, HEK293T cells were seeded, transfected with the library and the 3rd generation lentiviral packaging plasmids. Viral vectors were concentrated, titrated and the library was sequence verified by next-generation sequencing.

Transduction of Cells and Optional Selection

To generate a T cell population expressing an anti-BCMA CAR library and controls, T cells were isolated by immunoaffinity-based enrichment from leukapheresis samples from human donor subjects. T cells were activated with TransAct™ (Miltenyi Biotech™) in the presence of IL-2 (Miltenyi Biotech™) and transduced with the lentiviral library at an MOI of 0.2. After transduction and expansion, transduced cells were selected with anti-CD34 microbeads (Miltenyi Biotech™) as a truncated CD34 marker was co-expressed with the CAR.

T Cell Activation

Human T cells expressing the CAR library were co-cultured for 6 h with adherent wild-type HeLa (non-activated cells) or BCMA-expressing HeLa cells. Alternatively, non-tissue culture treated plates were coated overnight with recombinant hBCMA or control peptide. The next day plates were washed with PBS and blocked for two hours with blocking buffer. Subsequently, CAR cell library was activated with the recombinant hBCMA, for 6 h. After activation, cells from the supernatant were gently harvested in order to avoid contamination with adherent HeLa cells.

Single Cell RNA Sequencing

Single-cell suspensions were loaded onto a Chromium Single Cell Chip™ (10× Genomics™) according to the manufacturer's instructions. Cells were loaded into Single Cell chips and partitioned into Gel Bead In-Emulsions in a Chromium Controller™ (10× Genomics™) according to manufacturer instructions. The single cell RNA libraries were prepared according to the 10× Genomics™ Chromium Single Cell™ 3’ User Guide and sequenced on a HiSeg4000 ™ (Illumina™). Reads from single-cell RNA sequencing experiments were aligned to the GRCh38 genome and collapsed into unique molecular identifier (UMI) counts using the 10× Genomics™ Cell Ranger software. Samples were quality controlled to have sufficient numbers of genes detected, a high percentage of reads mapped to the respective genome, and sufficient number of cells detected.

Linking of scFv Sequence to Cell Barcode by Long Read Sequencing

In order to link the single cell RNA-seq information to the structure and sequences of the CAR library, long-read sequencing was performed. To this end, the barcoded single cell library was PCR amplified with a biotinylated forward primer, specific to the viral promoter region and a reverse U5 primer, specific for the 10× Genomics™ library. PCR product was cleaned up using magnetic streptavidin beads. A second nested PCR was performed to further enrich for specific PCR products, using a nested vector specific forward primer and the U5 reverse primer. PCR products were purified and prepared with the Ligation Sequencing Kit™ (Oxford Nanopore™) and sequenced on a Minion sequencing device according to manufacturers instructions.

### EXAMPLES

### Example 1

A synthetic human scFv phage library was panned against recombinant human BCMA protein (R&D biosystems™). To this end, the library was grown to log phase, and then rescued with M13KO7 helper phage (Antibody Design Lab™, PH010L) before being amplified overnight at 32° C. in a shaker. The phage library was subsequently precipitated with PEG/NaCl, re-suspended in PBS and stored at −80° C. Protein G coated magnetic beads were coated with 5 pg hBCMA-Fc or TNFRSF13B-Fc recombinant protein in PBS and subsequently blocked in PBS+BSA. Phage particles were incubated for 30 minutes with TNFRSF13B magnetic particles. Subsequently, magnetic particles were pelleted and the supernatant was incubated for 1 h with BCMA coated magnetic beads under rotation. After incubation, unbound and non-specifically bound phages were washed away by rinsing the beads with PBST. Bound phages were eluted by 100 mM triethylamine (TEA), and the eluate was neutralized by 1 M Tris-HCl (pH 7.4). The eluate was then used to infect exponentially growing E. coli TGI cells. The panning was repeated for an additional two to four cycles.

### Example 2

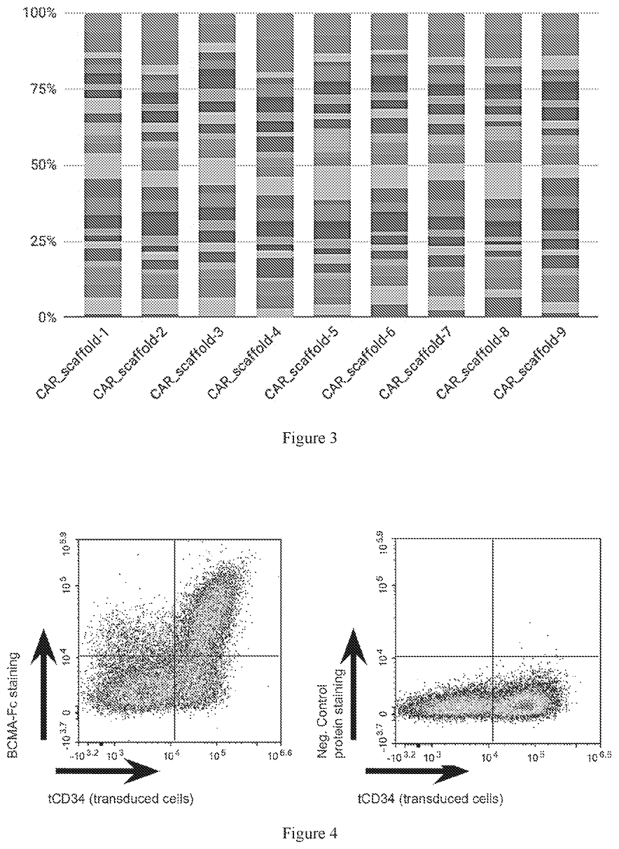

After the last panning step, scFv sequences were PCR amplified from the eluted phages or the isolated plasmids using Q5 DNA polymerase (NEB) and scFv-specific forward and reverse primers. Resulting amplicon was cleaned by PCR clean-up and 2 pg PCR product were digested with Kpn2I and XmaJI (Thermo Fisher™) for 2 h at 37° C. In parallel, a CAR scaffold library, consisting of pooled plasmids containing different CAR scaffolds with variation in hinge domain, transmembrane domain and intracellular signalling domains, was digested with the upstream restriction sites XmaJI and Kpn21. After 2 h incubation the CAR scaffold library and the scFv amplicons were cleaned by PCR clean-up columns and ligated using T4 DNA ligase (Thermo Fisher™) for 12 h over-night. Resulting CAR-library was electroporated into electrocompetent bacteria and grown overnight at 30° C., followed by Plasmid maxi-prep isolation. The CAR sequences from the resulting plasmid library were briefly PCR amplified and sequenced on an Oxford Nanopore Technology™ Min ION™, using the amplicon sequencing kit (FIG. 3).

### Example 3

In order to assess if the CAR library was functionally expressed and recognizing BCMA, the CAR library against BCMA was used to produce lentiviral vector particles and was then transduced into primary human T cells. The resulting CAR-T cell library was expanded for six more days after transduction and assessed for BCMA binding. BCMA-CAR-T cell library cells were stained with BCMA-Fc fusion protein (FIG. 4, left) (TNFRSF17-Fc, R&D systems) or a negative control protein (TNFRSF13B-Fc, R&D systems) (FIG. 4 right). In a second step, cells were stained with PE-conjugated anti-Fc and APC-conjugated anti-CD34 antibody to detect transduced cells. The expression of CARs on the cell surface was assessed.

### Example 4

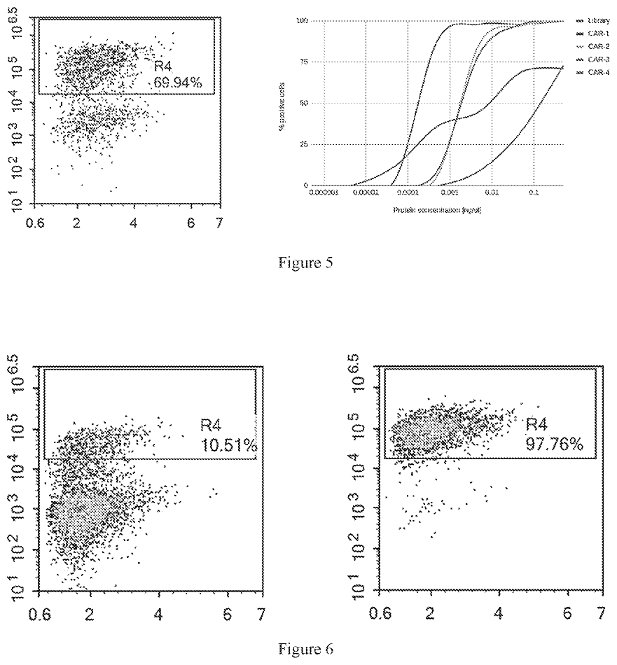

In order to assess if the CAR library contained CARs with low affinity/avidity towards BCMA, comparable to clinically tested CARs, the CAR library against BCMA was transduced into primary human T cells and titrated with BCMA-Fc protein in order to determine affinity/avidity. rBCMA-Fc protein was titrated on the cells in order to determine frequency of CARs with a given affinity/avidity on a population scale. In a second step, cells were stained with a fixed concentration of anti-Fc PE-conjugated antibody and frequency of positive cells was assessed by flow cytometry (FIG. 5). Four different CARs were expressed as a single CAR and were used as comparison to determine affinity/avidity relative to the rest of the library. Single CARs showed very defined affinity/avidity concentrations at which they were able to bind to BCMA (FIG. 5, right), however below that level they were no longer able to bind BCMA. In comparison, the BCMA CAR-T library showed a very spread out affinity/avidity, indicating multiple CARs with variable affinity and/or affinity being present. Next, BCMA library was incubated with 0.0001 ng/μl BCMA-Fc, followed by staining with anti-Fc PE antibody (Thermo Fisher™) (FIG. 6 left). Cells were subsequently incubated with anti-PE microbeads (Miltenyi™) and enriched through LS columns (Miltenyi™). Positively enriched cells were assessed by flow cytometry (FIG. 6 right). Subsequently, Genomic DNA was isolated and CAR sequences were amplified by PCR using Q5 DNA polymerase (NEB) and sequenced on a MinION™ sequencing device in order to assess enriched CAR sequences.

### Example 5

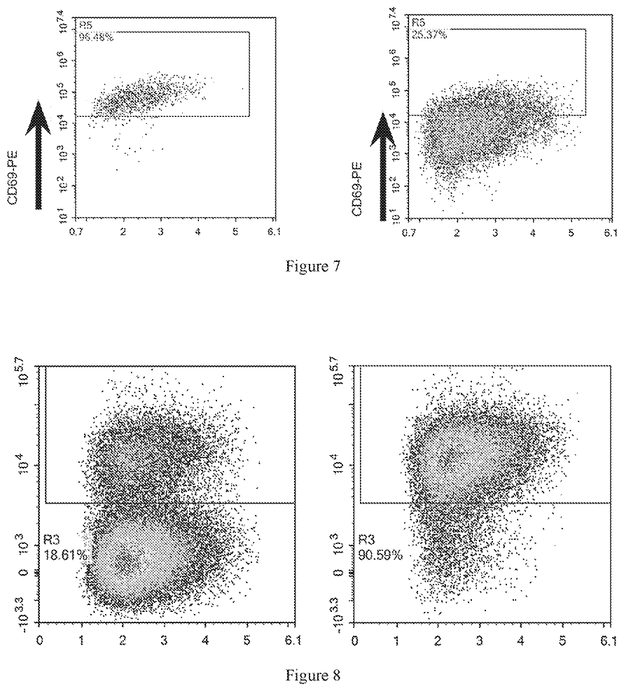

A CAR-T cell library, reactive against BCMA, was generated and exposed to BCMA-expressing HeLa cell line. After 24 h, activated CAR-T cells were sorted for CD69-positivity by flow cytometry or by magnetic bead selection (FIG. 7) (anti-CD69 microbeads (Miltenyi™)) and enrichment through LS columns (Miltenyi™). Similarly, CAR-T library cells were labelled with CFSE dye and sorted by flow cytometry for activated, highly dividing cells. Similarly, CAR-T library cells producing IFN-gamma were isolated using the human IFN-γ Secretion Assay and Enrichment Kit (Miltenyi Biotec). Genomic DNA from resulting enriched CAR-T cells was isolated and CAR sequences were amplified by PCR using Q5 DNA polymerase (NEB) and sequenced on a MinION™ sequencing device in order to assess enriched CAR sequences.

### Example 6

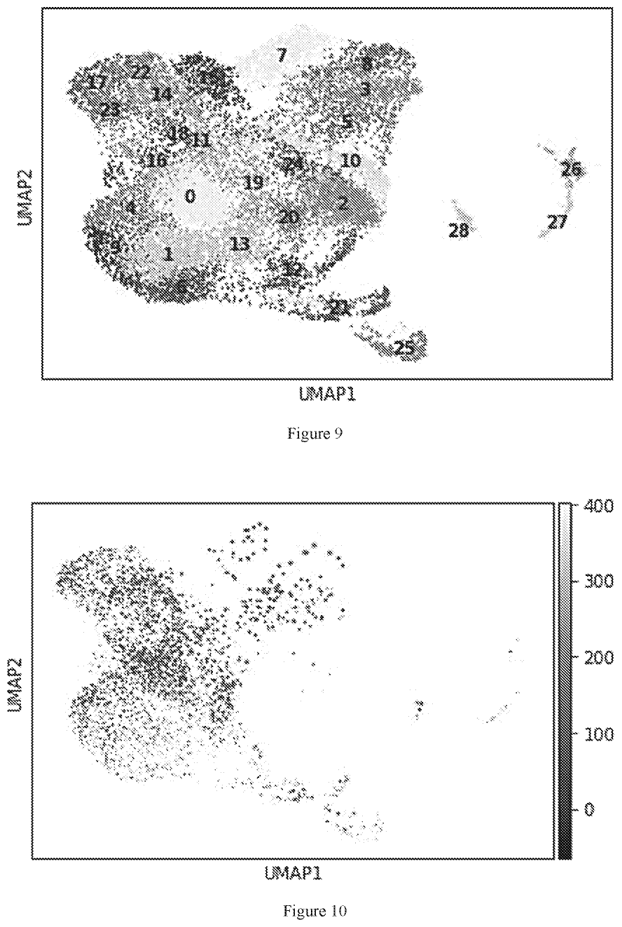

A CAR-T cell library was produced by activation of PBMCs with TransAct™ (Miltenyi™) in the presence of human IL-2 (100 IU/ml) and transduced at day 2 at a low MOI of 0.3 with the lentiviral CAR library against BCMA. The next day cells were washed and further expanded until day 8 of the process. Transduction was assessed by flow cytometry (FIG. 8, left) and transduced cells were enriched through CD34 microbeads (Miltenyi™). Purity was assessed by flow cytometry (FIG. 8 right). The CAR-T-cell library was co-cultured with BCMA-positive cells (RPMI-8226, U266, OPM-2 or HeLa-BCMA) or control cells (HeLa wild-type). After 6 h activation, CAR-T cells were re-enriched through LS columns (Miltenyi™) (FIG. 8 right) in order to deplete cancer cells and dead cells that impede single cell sequencing analysis. CAR-T cell libraries from different donors were prepared for 10× genomics single cell gene expression analysis (10× genomics 3′ sequencing kit V3 or Single Cell 5′ Kit v2) and sequenced on a NovaSeg™ 6000 (Illumina™). Cell ranger™ software (10× Genomics™) was used for downstream processing and alignment of reads. Dimensional reduction (UMAP) is shown and clusters were assigned to the different groups (FIG. 9). From the single cell full-length cDNA library the CAR sequence was amplified using CAR-specific primers and the Illumina™ read 1 primer to identify the relation between CAR-sequence and 10× Genomics™ single cell sequencing barcode. The −3.5 kB amplicons were sequenced on an Oxford Nanopore™ Min ION™ device using the amplicon sequencing kit.

### Example 7

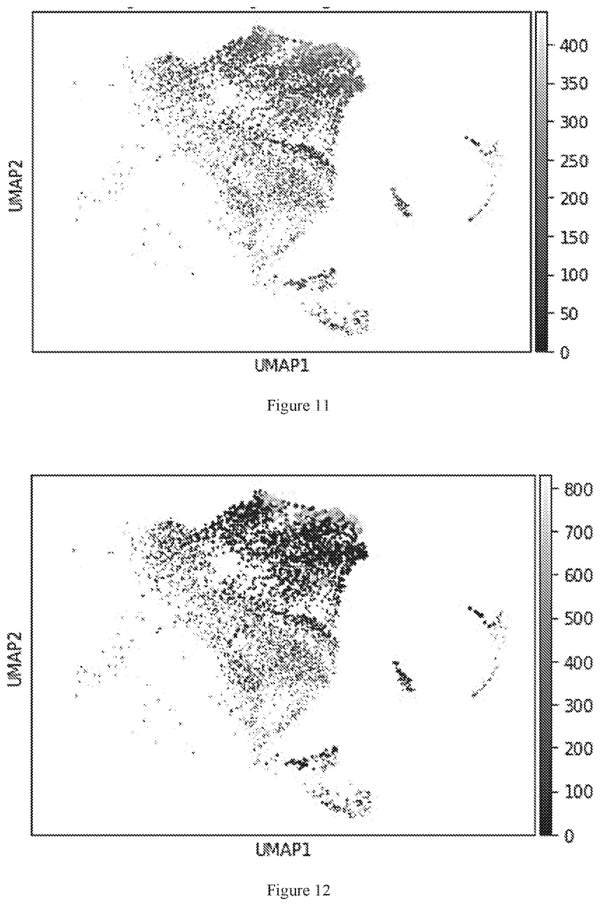

Single cell data were analysed and different gene signatures were defined. Machine learning methods were used to define functional clusters and signatures such as tonic CAR activation in the absence of target cells (FIG. 10). CAR-T activation signature was defined based on known activation genes and degree of activation was assessed (FIG. 11). CAR sequence, corresponding to the 10× Genomics™ single cell sequencing barcodes, was identified for all CARs through long-read Oxford Nanopore™ sequencing. Sequences corresponding to the CAR-libraries and the corresponding 10× Genomics™ barcodes were amplified from cDNA library using a CAR-specific forward primer and the Illumina™ read 1 primer. A second nested PCR was performed and amplicons were prepared for sequencing on Oxford Nanopore™ MinION™ using the ligation sequencing kit (Oxford Nanopore Technologies™). CARs with high clinical overall response rate were identified (FIG. 12) and a gene cluster was assigned in UMAP. CAR sequences were ranked according to this gene cluster and new anti-BCMA sequences were identified.

Although particular embodiments of the invention have been disclosed herein in detail, this has been done by way of example and for the purposes of illustration only. The aforementioned embodiments are not intended to be limiting with respect to the scope of the invention. It is contemplated by the inventors that various substitutions, alterations, and modifications may be made to the invention without departing from the spirit and scope of the invention.

1. A method for high-throughput screening of a chimeric antigen receptor (CAR) cell library, the method comprising the steps of:
a) providing:
i) a recognition sequence library, wherein the recognition sequence library comprises a one or more recognition sequences, wherein each recognition sequence encodes one or more antigen binding domains;
ii) a hinge region sequence library, wherein the hinge region sequence library comprises one or more hinge region sequences, wherein each hinge region sequence encodes a CAR hinge region domain;
iii) a transmembrane sequence library, wherein the transmembrane sequence library comprises one or more transmembrane sequences, wherein each transmembrane sequence encodes a CAR transmembrane domain; and
iv) an intracellular domain sequence library, wherein the intracellular domain sequence library comprises one or more intracellular domain sequences, wherein each intracellular domain sequence encodes one or more intracellular domains;
b) preparing a CAR library from the combination of:
i) a recognition sequence from the recognition sequence library;
ii) a hinge region sequence from the hinge region sequence library;
iii) a transmembrane sequence from the transmembrane sequence library; and
iv) an intracellular domain sequence from the intracellular domain sequence library; wherein the CAR library comprises a plurality of CAR sequences, each of the CAR sequences comprising one recognition sequence, one hinge region sequence, one transmembrane domain sequence and one intracellular domain sequence, and wherein each CAR sequence is a single continuous sequence that encodes a CAR;
c) preparing a CAR-cell library by introduction to, and expression of, the plurality of CAR sequences of the CAR library in one or more cells or a cell line so a plurality of CARs encoded by the plurality of CAR sequences are expressed on a surface of the one or more cells or a cell line;
d) screening the CAR-cell library in an assay that reports at least one function of each member of the CAR-cell library;
e) evaluating the at least one function of each member of the CAR-cell library;
f) obtaining one or more sequences, or a part thereof, of one or more of the plurality of CARs expressed in the CAR-cell library and linking the obtained sequence(s), or the part thereof, to the at least one function of one or more members of the CAR-cell library;
g) identifying and selecting the or each sequence that is responsible for a desired function of a member of the CAR-cell library.

2. The method of claim 1, wherein in step (0 the sequence, or part thereof, of one or more of the plurality of CARs in the cells in the CAR-cell library are identified and linked to the function of an individual cell within the CAR-cell library.

3. The method of claim 1 or claim 2, wherein the CAR-cell library has at least two points of diversity in the structure of each CAR represented therein.

4. The method of claim 3, wherein the points of diversity are selected from the group consisting of: one or more antigen binding domains; the hinge domain; the transmembrane domain; and the intracellular domain and combinations thereof.

5. The method of any one of claims 1 to 4, wherein the CAR library comprises one or more CAR vectors, each CAR vector encoding a CAR.

6. The method of claim 5, wherein introduction of the or each CAR in the CAR library in step (c) comprises transfection, transduction or electroporation of the CAR vector into the one or more cells or a cell line of the CAR-cell library.

7. The method of any one of claims 1 to 6, wherein the method is used for high-throughput screening of more than 10 CAR expressed in the CAR-cell library.

8. The method of any one of claims 1 to 7, wherein each member of the CAR-cell library comprises a unique CAR.

9. The method of any one of claims 1 to 8, wherein the at least one function of each member of the CAR-cell library is selected from the group consisting of: affinity binding to a target; downstream signalling of the CAR intracellular domain; modulation of protein and/or RNA expression.

10. The method of claim 9, wherein the function in step (d) is a measurable activation of the cells in the assay of step (d), to provide activated cells.

11. The method of claim 10, wherein activated cells may be identified based on up-regulation of: activation markers; cytokines; and/or introduced reporters driving expression of a reporter gene.

12. The method of claim 11, wherein activated cells may be identified by a method selected from: FACS sorting and magnetic enrichment or a combination thereof.

13. The method of claim 12, wherein the CAR sequence of an activated cell is identified using one or more methods selected from the group consisting of: long-read sequencing; next generation sequencing (NGS), and Sanger sequencing.

14. The method of any one of claim 9, wherein the function in step (d) comprises modulation of the RNA expression of the cell in the CAR-cell library.

15. The method of claim 14, wherein evaluation of the modulation in gene expression comprises analysis of RNA expression of one or more genes.

16. The method of claim 15, wherein the analysis of RNA expression of one or more genes uses single cell RNA expression measurement techniques.

17. The method of claim 16, wherein reporting of the modulation in gene expression comprises comparison of single cell RNA expression data of screened cells against the same, control cells that have not been subject to screening.

18. The method of claim 17, wherein the comparison uses RNA-seq techniques to analyse the cellular transcriptome of each cell in the CAR-cell library.

19. The method of any one of claims 14 to 18, wherein expressed mRNA of each cell in the CAR-cell library is associated with a unique identifier.

20. The method of claim 19, wherein mRNA of the CAR expressed in the cell is associated with the same unique identifier as the other mRNA from that cell such that the unique identifier may be used to associate the CAR to the mRNA expression of an individual cell.

21. The method of claim 19 or 20, wherein the unique identifier is a barcode sequence.

22. The method of claim 21, wherein in the barcode sequence is a DNA barcode sequence attached to a cDNA complimentary to the expressed RNA.

23. The method of any one of claims 19 to 21, wherein the unique identified allows linking of the sequence of the or each individual CAR, or part thereof, to the function of an individual member of the CAR-cell library in step (g).

24. The method of any one of claims 1 to 23, wherein the sequence may be obtained using one or more methods selected from the group consisting of: long-read sequencing; next generation sequencing (NGS) and Sanger sequencing.

25. The method of claim 24, wherein the one or more CARs of interest identified in step (g) provide novel CAR structures for a given target.

26. The method of claim 24, wherein the one or more CARs of interest identified in step (g) are used for the design of further iterations of the method of screening of claim 1.

27. The method of claim 26, wherein machine learning algorithms are used to identify and select CAR sequences or parts thereof for further iterations of screening,

28. The method of any one of claims 1 to 27, wherein one or more CAR sequences in the CAR library further comprises a co-stimulatory domain sequence.

29. The method of any one of claims 1 to 28, wherein the recognition sequence, the hinge region sequence, the transmembrane sequence. the intracellular domain sequence, and the CAR sequence are each a nucleic acid sequence.

30. The method of claim 29, wherein the nucleic acid sequence is a DNA sequence.

31. The method of any one of claims 1 to 30, wherein screening of the library is performed in a high-throughput manner selected from the group consisting of: simultaneously; in parallel; pooled: batchwise and any combination thereof.

32. The method of any one of claims 1 to 31, wherein at least steps (d) to (g) of the screening method may be completed within 24 hours.

33. The method of any one of claims 1 to 32, wherein in step (f), an individual sequence, or part thereof, is obtained and linked to the at least one function of an individual member of the CAR-cell library in which the individual sequence is expressed.

34. A method for high-throughput screening a chimeric antigen receptor (CAR) library, the method comprising the steps of:
a) providing a recognition sequence library, wherein the recognition sequence library comprises one or more recognition sequences, wherein each recognition sequence encodes for one or more antigen binding domains;
b) preparing a CAR library from the combination of a recognition sequence library and a CAR scaffold, wherein the CAR scaffold comprises a hinge region sequence, a transmembrane domain sequence and an intracellular domain sequence, wherein the CAR library comprises a plurality of CAR sequences, each of the CAR sequences comprising one of the one or more recognition sequences, the hinge region sequence, the transmembrane domain sequence and the intracellular domain sequence, and wherein each CAR sequence is a single continuous sequence that encodes a chimeric antigen receptor;
c) preparing a CAR-cell library wherein each CAR sequence of the CAR library is introduced to, and expressed as a CAR on the cell surface of, one or more cells or a cell line;
d) screening the CAR-cell library in an assay that reports at least one function of each member of the CAR-cell library;
e) evaluating the at least one function of each member of the CAR-cell library;
f) obtaining one or more sequences, or a part thereof, of one or more of the plurality of CARs expressed in the CAR-cell library and linking the obtained sequence(s), or the part thereof, to the at least one function of one or more members of the CAR-cell library;
g) identifying and selecting the or each sequence that is responsible for a desired function of a member of the CAR-cell library.

35. The method of claim 34, wherein the CAR scaffold is provided as a single continuous sequence for combination with the recognition sequence library.

36. The method of claim 34 or claim 35, wherein the CAR scaffold is assembled from the hinge region sequence, the transmembrane domain sequence and the intracellular domain sequence separately prior to or after combination with the recognition sequence library.

37. The method of claim 36, wherein the CAR scaffold is provided as separate components when combined with the recognition sequence, wherein the separate components are one or more sequences selected from the group consisting of: one or more hinge region sequences, one or more transmembrane domain sequences and one or more intracellular domain sequences.

38. The method of claim 37, wherein the recognition sequence is combined with the hinge region sequence prior to combination with other components to provide a single continuous sequence encoding for a complete CAR.

39. The method of claim 38, wherein step (b) comprises addition of further components of the CAR sequence in a sequential and/or parallel manner using individual sequences or libraries of a plurality of sequences, of components in an appropriate order.

40. The method of claim 39, wherein, addition of the components is after one or more of the components has been combined.

41. The method of any one of claims 34 to 40, wherein the CAR library comprises a plurality of CAR sequences, each sequence encoding for a CAR.

42. The method of claim 41, wherein each CAR sequence in the CAR library is formed of a recognition sequence and a CAR scaffold comprises a hinge region sequence, a transmembrane domain sequence and an intracellular domain sequence.

43. The method of any one of claims 34 to 42, wherein in step (f), an individual sequence, or part thereof, is obtained and linked to the at least one function of an individual member of the CAR-cell library in which the individual sequence is expressed.

44. A CAR library for use in the high-throughput screening method of any one of claims 1 to 43, wherein the CAR library comprises a plurality of single continuous sequences encoding for a CAR, the CAR comprising a recognition sequence from the recognition sequence library, a transmembrane domain sequence and an intracellular domain sequence, and wherein each CAR sequence is a single continuous sequence that encodes a chimeric antigen receptor.

45. The CAR library of claim 44, wherein the CAR library has at least two points of diversity in the sequence of each CAR represented therein.

46. The CAR library of claim 45, wherein the points of diversity are selected from the group consisting of: one or more antigen binding domains; the hinge domain; the transmembrane domain; and the intracellular domain.

47. The CAR library of any one of claims 44 to 46, wherein the one or more antigen binding domains comprises at least two or more antigen binding domains.

48. The CAR library of any one of claims 44 to 47, wherein the CAR library comprises sequences for more than 10 CARs.

49. The CAR library of any one of claims 44 to 48 wherein the CAR-library is provided in a format suitable for introduction into cells or a cell line to form a CAR-cell library.

49. AR library of claim 49, wherein each CAR sequence in the CAR library is provided as a vector suitable for introduction and/or expression of the CAR sequence into cells.

51. The CAR library of claim 50, wherein transfection is transduction or electroporation.

52. The CAR library of any one of claims 49 to 51, wherein the CAR sequences of the CAR-library are provided in a format selected from: plated with CAR sequences split into separate wells or compartments of a plate; pooled where more than one CAR sequence is in a single well or compartment; and combinations thereof.

53. A method of preparing a CAR library of any one of claims 44 to 52, wherein the method comprises steps (a) and (b) of claim 1 or steps (a) and (b) of claim 34.

54. A CAR-cell library for use in the high-throughput screening method of any one of claims 1 to 43, wherein the CAR-cell library comprises a plurality of cells, each cell having at least one CAR expressed on its surface, wherein each CAR comprises a recognition sequence domain, a hinge region domain, a transmembrane domain and an intracellular domain.

55. The CAR-cell library of claim 54, wherein the CAR-cell library has at least two points of diversity in the structure of each CAR represented therein.

56. The CAR-cell library of claim 55, wherein the points of diversity are selected from the group consisting of: one or more antigen binding domains; the hinge domain; the transmembrane domain; and the intracellular domain.

57. The CAR-cell library of any one of claims 54 to 56, wherein the CAR-cell library comprises more than 10 CARs.

58. The CAR-cell library of any one of claims 54 to 57, wherein a single CAR is expressed on the surface of a single cell.

59. The CAR-cell library of any one of claims 54 to 58, wherein the cell is a T-cell.

60. The CAR-cell library of claim 59, wherein the T-cell is a primary human T-cell.

61. The CAR-cell library of any one of claims 54 to 60 wherein the CAR-cell library is provided in a format suitable for screening.

62. The CAR-cell library of claim 61, wherein the cells of the CAR-cell library are provided in a format selected from: plated with one or more cells split into separate wells or compartments of a plate; pooled where more than one cell is in a single well or compartment of a plate; and combinations thereof.

63. A method of preparing a CAR-cell library of any one of claims 54 to 62, wherein the method comprises steps (a), (b) and (c) of claim 1 or steps (a), (b) and (c) of claim 34

64. A single cell CAR-cell library for use in the screening method of any one of claims 1 to 43, wherein the single cell CAR-cell library comprises a plurality of single cells, each single cell taken from a representative member of the CAR-cell library of any one of claims 54 to 63.

65. Use of the method of any one of claims 1 to 43 for the identification of CARs having a desired function.

66. Use of the CAR library of any one of claims 44 to 52 to prepare a CAR-cell library for screening.

67. Use of a CAR-cell-library of any one of claims 54 to 62 for screening the function of a plurality of CARs.

68. The use of claim 67, where the screening is performed in a high-throughput manner selected from the group consisting of: simultaneously, in parallel, pooled, batchwise.

69. The use of claim 67 or claim 68, wherein the plurality of CARs is more than 10 CARs.
